(19) United States
(12) Patent Application Publication
(10) Pub. No.: US 2002/0061869 A1
(43) Pub. Date: May 23, 2002

(54) Free insulin testosterone test

(76) Inventor: Edward M. Lichten, Southfield, MI (US)

Correspondence Address: SONNENSCHEIN NATH & ROSENTHAL, Wacker Drive Station, Sears Tower, P.O. Box 061080, Chicago, IL, 60606-1080, US

(21) Appl. No.: 09/898,770

(22) Filed: Jul. 2, 2001

Related U.S. Application Data
(63) Continuation-in-part of application No. 09/198,798, filed on Nov. 24, 1998.

(51) Int. Cl. A61K 31/56

(52) U.S. Cl. 514/178; 514/866

(57) ABSTRACT

The present invention is directed to a method of identifying a mammal with a hormone disorder, or identifying a mammal at risk of developing a hormone disorder. The method comprises determining the serum concentration of estrogen, testosterone, sex hormone-binding globulin, and insulin in the mammal, calculating their ratio, or F factor, and administering to the mammal a hormone disorder-effective amount of a therapeutic agent until a therapeutic F factor is achieved, or maintained in the mammal. The present invention is also directed to methods, kits, combinations, and compositions for treating, preventing or reducing the risk of developing a hormone disorder, or a disease or disorder associated with, or related to, a hormone disorder, such as cardiovascular disease.

### CROSS-REFERENCE TO RELATED APPLICATION

This is a continuation-in-part of U.S. patent application Ser. No. 09/198,798, entitled, “Use of Testosterone to Treat Impaired Glucose Tolerance and Insulin Resistance and Method of Screening for Insulin Resistance in Adult Onset Diabetes and Syndrome X,” filed Nov. 24 1998, the disclosure of which is expressly incorporated by reference.

### FIELD OF THE INVENTION

The present invention relates to a method of identifying a mammal with a hormone disorder, or a mammal at risk of developing a hormone disorder; and to methods, kits, combinations, and compositions for treating a mammal with a hormone disorder or a mammal at risk for developing a hormone disorder, or a disease or disorder associated with, or related to, a hormone disorder.

### BACKGROUND OF THE INVENTION

The cause and manifestation of cardiovascular disease is multifactorial. Many studies have sought to discover a common hormonal denominator to explain the predominance of coronary arterial disease in men. There is agreement among many authors that the presence of hypo-testosteronemia (low testosterone) (Phillips et al., 1994), hypoadrenalism (low dehydro-epiandrosterone sulfate) (Nafziger et al., 1991; Mitchell et al., 1994; and Newcomer et al., 1994) and/or increased sex hormone-binding globulin (Phillips et al., 1994) defines a state for men for increased risk of coronary artery disease and myocardial infarction. However, there is no such consensus for women.

Based on retrospective epidemiological studies and prospective clinical trials, the major cardiovascular disease risk factors reported in serum assays include cholesterol, insulin, and sex hormones. The sex hormones include, for example, estrogen and testosterone. In post-menopausal women, focus has been on the inter-correlation of three cardiovascular risk factors: sex hormone-binding globulin, insulin, and cardiovascular disease. In men, focus has been on total cholesterol/HDL-cholesterol. In men and women, the sex hormone-binding globulin is an independent risk factor of cardiovascular disease, independent of HDL cholesterol, triglycerides, apolipoprotein-B, and HDL-C/cholesterol ratio.

Serum concentration of the male sex hormone testosterone has a correlation to well-being in men. For example, levels of total and free serum testosterone concentrations have a correlation with hyperinsulinemia, insulin, and heart disease. Additionally, testosterone lowers cholesterol and normalizes abnormal electrocardiograms of patients. Testosterone can also improve diabetic retinopathy as well as lower the insulin requirements of diabetic patients and decrease the percentage of body fat. Administration of testosterone to men also decreases the risk factors for heart attack and low serum concentration of testosterone is also correlated with hypertension, obesity, and increased waist-to-hip ratio. There is also a drop of serum concentration of free testosterone of about 1.5% per year following puberty. While the total testosterone of a male does not drop drastically, the free testosterone, which is the biologically active testosterone, does drop with aging. In fact, a drop of free testosterone can occur as early as the early forties. Men with high testosterone levels live longer, healthier lives and maintain sexual potency. Testosterone also has the ability to stop the spread of breast cancer in females. Additionally, testosterone has a protective effect against autoimmune diseases.

The female sex hormones, estrogen and progesterone, are known to drop to very low levels after menopause. Several prestigious medical groups, including the American College of Physicians and the American College of Obstetricians and Gynecologists, have released position papers stating that post-menopausal women should seriously consider preventive estrogen/progesterone hormone replacement therapy for their benefit in reducing osteoporosis and heart disease, the major scourges of old age in women. Maintaining estrogen and progesterone levels has also been shown to improve a number of key risk factors for heart disease in post-menopausal women. Oral estrogen replacement therapy improves the risk factors for cardiovascular disease including plasma lipids and fibrinogen. The replacement of estrogen in menopausal women has been shown to reduce cardiovascular disease mortality by fifty percent. Yet, to date, researchers have been unable to correlate estrogen measurements with the presence of heart disease.

Many observational studies have elucidated a correlation between estrogen use and a decrease in cardiovascular disease. The Framingham study (Kannel W. B., et al., Ann. Intern. Med. 1976; 85(4):447-52) and Nurses' study (Stampfer M. J., et al., N. Engl. J Med. 1991; 325(11):756-62; Martin K. A., Freeman M. W. N. Engl. J. M. 1993; 328(15):1115-17) showed that women who used estrogen replacement therapy experienced dramatically less heart disease. However, measurements of random estradiol, total estrogen, and free estradiol have not shown correlation to cardiovascular risk parameters. And one of the directives of the Women's Health Initiative begun in 1992 as a ten-year $862 million government-funded project was to determine the role that estrogen might have in reducing cardiovascular risk in menopausal women.

In men, the Free Androgen Index test, used extensively in Europe, has shown an inverse relationship between free estradiol and sex hormone-binding globulin, and an inverse and logarithmic relationship between testosterone and sex hormone-binding globulin. The drop in testosterone is between 5 and 15 nmol/liter and mostly linear paralleling estradiol as sex hormone bonding globulin increases.

The sex hormone testosterone, by itself or in relative ratio with estrogen, may be a relative risk factor for cardiovascular disease. While exogenous testosterone may negate the benefits of estrogen replacement therapy, and metabolic states with high testosterone or testosterone precursors are associated with increased cardiovascular risk (Gorbach S. L., et al., Metabolism 1989; 38(11):1077-81). Studies by Haffner et al. did not confirm significant correlation for testosterone or estrogen/testosterone ratios; see, for example, Haffner S. M., et al., Metabolism 1992; 41(3):278-84. Also see, for example, Haffner S. M., et al., Arteriosclerosis 1989; 9(1):136-43. Additionally, see Haffner S. M., Valdex R. A., Am. J. Med. 1995; 98(1A):40S-47S. However, an indirect measurement of androgenicity, sex hormone-binding globulin, has been shown to be directly related to insulin resistance, an atherogenic lipid profile, impaired glucose tolerance, and heart disease; see, for example, Lindstedt G., et al., Diabetes 1991; 40(1):123-28. Also see, for example, Preziosi P., et al., J. Clin. Endocrinol. Metab. 1993; 76(2):283-87. Additionally, see, Sherif K., et al., Metabolism 1998; 47(1):70-74.

The structure and proposed function of sex hormone-binding globulin have been described and characterized; see, for example, Rosner et al., J. Steroid Biochem. Mol. Biol., Vol. 69:481-85 (1999). See also, for example, Petra, P. H., J. Steroid Biochem. Mol. Biol., Vol. 40:735-53 (1991). A variety of methods have been used to quantify the serum concentrations of sex hormone-binding globulin, including ammonium sulfate precipitation, gel filtration, equilibrium dialysis, dextran-coated charcoal, and radioimmunoassay (see, for example, Kahn et al., J. Clinical Endocrinology and Metabolism, Vol. 54:705-10 (1982)). The mean serum sex hormone level in healthy pre-menopausal women is about 84 nmol/L and the normal range is about 36 nmol/L to about 185 nmol/L. Serum sex hormone-binding globulin levels are known to be elevated in women treated with oral estrogens, estrogen-containing oral contraceptives, clomiphene, tamoxifen, raloxifene, phenytoin, and sodium valproate, as well as in women who are pregnant, hyperthyroid, have chronic liver disease and HIV infection. See, for example, Bond et al., Acta. Obstet. Gynecol. Scand., Vol. 66:255-62 (1987).

Insulin resistance is an essential component in the malfunction of the cell. The movement of glucose from the blood stream into the cell where it is used for energy is the basis for cellular homeostasis. Recent discoveries have identified three separate components to the process termed “insulin resistance.” These separate components are hyperglycemia (elevated serum levels of glucose), hyperinsulinemia (elevated serum levels of insulin), and elevated tissue levels of glycogen and glucose. When an individual has either hyperinsulinemia or elevated tissue levels of glycogen and glucose, the term “insulin resistant” is applied, because the addition of insulin does not correct these individual components.

Insulin plays an essential role in the body function. It was originally noted in diabetes mellitus because the absence of insulin often resulted in death of the individual. Banting and Best (1922) extracted insulin from the pancreas and demonstrated its usefulness in diabetic animals. Prepared in crystalline form by Abel (1926), the establishment of the amino acid sequence by Sanger (1960), the complete synthesis of the hormone by Katsoyannis (1966) and the development of various preparations with different half lives completes the historical perspective of insulin. See Randall H. Travis and George Sayers, The Pharamacological Basis of Therapeutics, Ch. 71 Insulin. Also see, Oral Hypoglycemic Drugs 1581-1603 (Louis S. Goodman and Alfred Gilman eds., 4th ed. 1970).

Insulin's mode of action on carbohydrate, protein, and fat metabolism remains the subject of intense investigation since its discovery severity years ago, for diabetes is identified by hyperglycemia, elevated glucose levels in the blood. In juvenile onset diabetes, destruction of the beta cells of islets of Langerhans of the pancreas creates an insulin deficiency, which prevents glucose uptake by the cells. But in adult onset diabetes, representing 90% of all diabetes, these patients demonstrate an increase in insulin production. This represents a cellular defect so that normal levels of insulin are not effective in clearing glucose from the blood stream. The condition in which the body produces an excess of insulin is referred to as “insulin resistance.”

Levine (1949) and associates made a notable contribution when they developed the concept that the cell membrane, under the influence of insulin, regulates glucose utilization by determining the rate at which glucose passes from extracellular to intracellular fluid, and thereby affects the rate of oxidation and the conversion to glycogen. Action of insulin at the membrane site explains many, but not all, of the varied effects of the hormone on intermediary metabolism. Current work suggests that insulin has multiple loci of action such as on the enzyme system that promotes the conversion of glucose to glycogen, and inhibits the mobilization of fat.

Travis and G. Sayers stated that “the explanation for the fact that large numbers of diabetics required much more insulin than was estimated to be secreted by the pancreas of a normal subject was sought in terms of abnormalities in the activities of the pituitary or the adrenal cortex” (Travis and Sayers, 1970).

Although insulin replacement in adult onset diabetes is the treatment prescribed, it does not prevent the development of accelerated system disease. The etiology of the characteristic defects in the cardiovascular system of the diabetic patient (e.g., retinopathy, atherosclerosis) remains unexplained. Their onset and progress do not appear to be dramatically influenced by treatment with insulin. Intense hypoglycemia can result in insulin coma, hypoglycemic convulsions and irreversible damage to the brain (Travis and Sayers, 1970). On the other hand, hyperglycemic tissue states induce cataracts, macular degeneration, obesity, and increased mortality risks from the deposits of glucose converted into lipid material within the cells. However, to date, Traverse and Sayers (1970) state that insulin is the most effective drug therapy for states of hyperglycemia.

Elevated levels of insulin are also associated with a number of abnormal states of which only one is diabetes mellitus. Since high levels of insulin block bile breakdown of fat deposits and encourage the breakdown of muscle, high insulin levels are a factor in obesity and muscle wasting. Hyperinsulinemia correlates with an increased incidence of cardiovascular disease in both men and women. Hyperinsulinemia also depresses bile normal production of anabolic steroids including testosterone, dehydro-epiandrosterone sulfate, and growth hormone. Hyperinsulinemia also contributes to the further worsening of insulin resistance, in part, by its ability to cause increased deposits of glucose/glycogen within the cell.

In regards to the treatment of diabetes, a number of other pharmaceutical products have been shown to lower the first characteristic finding of insulin resistance, elevated serum glucose (hyperglycemia). Sulfonamide was discovered by Janbon and coworkers (1942) to induce hypoglycemia. Loubatieres (1957) discovered that the compound had no effect on the pancreatized animal; in normal animals it increased the secretion of insulin from the pancreas. Franke and Fuchs (1955) found that carbutamide and tolbutamide, antibiotic and its derivatives, lowered blood sugar levels. These are the sulfonylureas.

Watanabe (1918) found that another group of compounds, the biguanides, lowers blood glucose levels. Although diguanides (Synthalin A) and guanidine derivatives were found too toxic for therapeutic use, phenformin (Ungar, 1957) was found useful with an acceptable level of toxicity, so it is used extensively today.

Other compounds show hypoglycemic potential but are not used because of their toxicity in effective doses. Salicylates lower blood glucose when given in large doses. Their hypoglycemic activity, of unknown mechanism, is too weak to justify their use in diabetes.

Travis and Sayers stated that the sulfonylureas represented a most significant contribution to the treatment of the diabetic patient over forty years of age with stable and mild diabetes. These agents have the decided advantage over insulin of being effective by the oral route. They are ineffective in the unstable diabetic and in the management of any type of diabetic during acute situations of fever, trauma, or surgery. It is very doubtful, however, that the sulfonylureas reduce the incidence of diabetic complications although they have been of great benefit in the treatment of hundreds of thousands of diabetic subjects.

Sulfonylureas should be used with caution in patients with impairment of hepatic or renal function. They are not recommended for use in patients with hepatic or renal insufficiency because of the important role of the liver in the metabolism of sulfonylureas and of the kidney in the excretion of the drugs and their metabolites. Intolerance to alcohol reminiscent of the disulfiram reaction has occurred occasionally in patients. These drugs are effective (for lowering serum glucose) in adult onset diabetes in whom less than 20-40 units are needed to maintain control.

Diabetes of prolonged duration seems less amenable to sulfonylureas. Onset before thirty years of age, and with unstable ketoacidosis, these patients require insulin and attempts to control them with oral therapy are dangerous and doomed to failure. Deaths from acidosis and dehydration have occurred in patients with unstable ketotic diabetes in whom regulation was attempted with sulfonylureas.

Phenformin, in combination with insulin, may develop ketonuria even with normal blood glucose levels. The cause is unknown and phenformin should be discontinued. Diabetics with severe renal insufficiency or congestive heart failure are not suitable candidates for oral hypoglycemic therapy (Travis and Sayers, 1970).

Troglitazone (Warner Lambert Co.) is the first antihyperglycemic agent which acts primarily by decreasing insulin resistance. It improves sensitivity to insulin in muscle and adipose tissue and inhibits hepatic glyconeogenesis. It is not chemically related to either the sulfonylureas, the biguanides or the alpha-glucosidase inhibitors.

Troglitazone is a thiazolidinedione anti-diabetic agent that lowers blood glucose by improving target cell response to insulin. It has a unique mechanism of action that is dependent on the presence of insulin for activity. Troglitazone decreases hepatic glucose output and increases insulin-dependent glucose disposal in skeletal muscle. Its mechanism of action is thought to involve binding to nuclear receptors (PPAR) that regulate the transcription of a number of lipid responsive genes critical for the control of glucose and lipid metabolism. Unlike sulfonylureas, troglitazone is not an insulin secrelagogue.

In animal models of diabetes, troglitazone reduces the hyperglycemia, hyperinsulinemia, and hypertriglyceridemia characteristic of insulin-resistant states such as type II (adult onset) diabetes. Plasma lactate and ketone body formation are also decreased. The metabolic changes produced by troglitazone result from the increased responsiveness of insulin-dependent tissues and are observed in numerous animal models of insulin resistance. Treatment with troglitazone did not affect pancreatic weight, islet number or glucagon content, but did increase regulation of the pancreatic beta cells in rodent models of insulin resistance.

Since troglitazone enhances the effects of circulating insulin (by decreasing insulin resistance), it does not lower blood glucose in animal models that lack endogenous insulin.

Since medical experience has demonstrated that the reduction of serum glucose in type II (adult onset) diabetes corrects ketoacidosis but does not prevent the other aspects of the disease (Travis and Sayers, 1970), effective treatment for diabetes relies on correction of both hyperinsulinemia and increased concentration of cellular glycogen. However, troglitazone, the only product recognized to lower insulin levels and the levels of cellular glycogen, has a number of potent and life threatening side effects. As reported to date, the side effects include abnormal liver function tests, jaundice, drop in white blood cell count and drop in hemoglobin which may persist for two years. It is recommended not to be used in combination with oral contraceptives, terfenadine, and choleslyramine, because it lowers the effective blood level of these drugs by up to 30 percent.

In animal studies, troglitazone was administered daily for 104 weeks to male and female rats in various doses. In female rats, there was a statistically significant increase in sarcomatous tumors at the high (200 mg/leg) dose (47-fold greater than estimated human exposure of parent compound). However these findings are of unknown clinical relevance as this dose was associated with excessive mortality and is considered to have surpassed the maximum tolerance dose (Physicians' Desk Reference 1998).

Because of this, the Physicians' Desk Reference notes that troglitazone is only indicated for use with type II diabetes currently on insulin therapy whose hyperglycemia is inadequately controlled (HbA_{tc},>8.5%) despite insulin therapy of over 30 units per day given as multiple injections.

But impaired glucose tolerance, hyperglycemia and insulin resistance are now seen as etiologic elements to much more than adult onset diabetes mellitus. Reaven (1988) defined Syndrome X as the constellation of various components: insulin resistance, glucose intolerance, hyperinsulinemia, increased VLDL triglyceride, decreased HDL cholesterol, and hypertension. Kaplan (1989) noted the coexistence of upper body (central) obesity, glucose intolerance, hypertriglyceridemia, and hypertension. Black (1996) added coronary artery heart disease to this syndrome. Therefore, by the consensus of these and other researchers, the constellation of laboratory findings of insulin resistance is the underlying or consistent component of diabetes, obesity, hypertension, dyslipidemia and heart disease. Insulin resistance is the key element of Syndrome X. Syndrome X affects 70-80 million people, almost one-third of Americans, is linked to obesity and weight gain, is associated with diabetes, is associated with high blood pressure, is a common factor in cardiovascular diseases and stroke, and is a primary cause of lowered metabolism and fatigue.

For descriptive purposes, all medical conditions with insulin resistance, including adult onset diabetes, will be included under the designation of Syndrome X. For the purpose of defining the measured laboratory components of insulin resistance, references are made to (1) hyperglycemia, (2) hyperinsulinemia and (3) increased tissue glycogen and their respective laboratory tests: (a) fasting serum glucose, (b) fasting serum insulin and (c) serum elevated hemoglobin A_{1c} (HbA_{1c}).

Because normal insulin metabolism is necessary for good health and to optimize delivery of many nutrients, interest in this area continues to increase within both the medical and pharmaceutical communities. Doctors and scientists all over the world are starting to see that many seemingly unrelated diseases are in fact linked to a malfunction in insulin and/or blood-sugar metabolism. Insulin's primary role is to lower blood-sugar levels by transporting carbohydrate energy (glucose) into and out of muscle and liver cells.

Secondarily, insulin helps pull amino acids into the cells, turns on protein synthesis and promotes fatty acid/triglyceride storage. Problems with the body's ability to regulate blood glucose crop up if the cells don't readily accept blood sugar or if insulin doesn't properly bind to its glucose transport receptors. When normal amounts of insulin do not reduce blood sugar after meals, the body secretes more and more insulin until serum glucose levels fall, sometimes too far. For example, the urge to snack at around 4 p.m. and 9 p.m. is typically tied to wild fluctuations in blood sugar levels. This is not to be confused with symptoms of either a potential diseased state or full-blown insulin resistance.

Insulin resistance most likely begins as a genetic predisposition that becomes manifested by the over-consumption of simple and refined carbohydrates, the lack of adequate nutrients combined with being sedentary (Grimditch, 1988). Consuming too many processed simple carbohydrates coupled with inadequate nutrient intake are common shortcomings of the typical American diet, and, of course, the American lack of adequate exercise. Conversely, and not surprisingly, diets and nutrients that reduce the amount of insulin required by the body also reduce the tendency toward excessive production of insulin (Murray, 1991). Exercise has also been shown to be an excellent modality for improving insulin sensitivity.

Well-known for its ability to store glucose in muscle and increase protein synthesis and muscle mass, insulin has only recently been recognized as the primary anabolic hormone produced in the body. Some researchers believe insulin is in fact more important to lean muscle tissue than the better-known anabolic hormones testosterone and growth hormone. Unfortunately, insulin resistant individuals also suffer from a drop in testosterone and growth hormone. Out-of-control insulin metabolism tends to make one gain body fat and catabolize muscle. With increased insulin resistance, both the number of calories stored as fat and the amount of fat produced by the liver from carbohydrates gets worse.

Because of the impaired ability of muscle to release glucose from the muscle, these individuals experience fatigue and a decreased ability to exercise. The problem of insulin resistance potentiates and accelerates the development of the various disease states of Syndrome X.

Proper insulin function is of paramount importance to those dealing with the constellation of symptoms associated with improperly functioning insulin metabolism. In terms of nutrition, over-consuming any of the macro nutrients (and particularly simple carbohydrates) should be avoided. As for supplements, micro-nutrients such as vitamins C and E, magnesium, omega-3 fatty acids, chromium and vanadyl sulfate have been shown to improve insulin sensitivity. (Murray, 1991). Diabetics, people with Syndrome X, and athletes who want to improve their insulin metabolism/sensitivity might choose to include these nutrients in their diets. And of course, regular exercise improves insulin sensitivity.

For all individuals, understanding insulin metabolism and the damage caused by too much glucose and insulin is essential for good health. At every age, one wants to be gaining muscle, losing body fat and using diet and those therapies that will prevent the spiral into insulin resistance. Avoiding Syndrome X and the many diseases associated with it means regular exercise, adequate and correct nutrient intake, shunning health-degrading substances and using some common sense. For physicians and health professionals, closer attention to prevention and early diagnosis will simplify treatment and minimize the manifestation of diseases associated with Syndrome X.

Although various dosages of testosterone have been used in the treatment of medical conditions, and there are at least three observational references attesting to the positive effects of testosterone on diabetes (Moppet and Einfeldt 1984, Carruthers 1996, Mauriello et al. 1997), there exists no previous documentation in either adult diabetes mellitus or any other disease associated with Syndrome X of the methodology and effects of using these three professed components to effect treatment: (1) pre-selection of patients based on the testosterone/derivatives ratio testing, (2) testosterone dosing schedule, based on reaching specific serum concentration of testosterone and/or its derivatives and (3) documentation of the reversal of all three components of insulin resistance: hyperglycemia, hyperinsulinemia, and abnormal glycosylated hemoglobin A_{1c} levels with testosterone and/or its derivatives.

Previous information on the relationship of sex hormone-binding globulin and disease is confusing. Post-menopausal women have a decreased level of sex hormone-binding globulin; see Berglund et al., 1996. Also see, for example, Bhasin et al., 1996. Yet, decreased sex hormone-binding globulin has predicted the development of non-insulin dependent diabetes mellitus in two populations; see Birkeland et al., 1993. Also see, for example, Black, 1996. Haffner et al., (1996) stated that insulin excess does not change the sex hormone-binding globulin but changes in the sex hormone-binding globulin change insulin levels; see also, Bhasin et al., 1996. Pasquali and Nestler disagreed, however, finding that diazoxide administration as an insulin blocking agent resulted in increased sex hormone-binding globulin (Faix et al., 1993; Franke and Fuchs, 1955).

A number of reports confirm the inverse relationship between sex hormone-binding globulin and insulin. Hyperinsulinemia and insulin resistance have been related to a decreased level of sex hormone-binding globulin in post-menopausal women (Soler et al., 1989; Haffner et al., 1993). Haffner states that “the alteration in sex hormone, rather than a direct effect of insulin” (1993) causes changes in lipid metabolism.

For these and other reasons, therefore, it would be a difficult but much desired advance in the art to provide an effective method to identify a mammal with a hormone disorder or a mammal at risk of developing a hormone disorder. In particular, it would be a difficult but much desired advance in the art to provide a method of identifying a mammal with a hormone disorder associated with, or related to, a disease or disorder, such as a cardiovascular disease, in the mammal, or to identify a mammal at risk of developing a hormone disorder that may lead to a disease or disorder, such as a cardiovascular disease, in the mammal. Also, it would be a difficult but much desired advance in the art to provide effective methods, kits, combinations, and compositions for treating, preventing or reducing the risk of developing a hormone disorder, or the symptoms associated with the hormone disorder, in a mammal in need thereof.

### SUMMARY OF THE INVENTION

The preceding problems are solved and a technical advance is achieved by the present invention. The present invention relates to a method of identifying a mammal with a hormone disorder, or a mammal at risk of developing a hormone disorder; and to methods, kits, combinations, and compositions for treating a mammal with a hormone disorder or a mammal at risk for developing a hormone disorder. In particular, the method comprises determining the serum concentration of the hormones estrogen, testosterone, sex hormone-binding globulin, and insulin in the mammal, and calculating the ratio of these hormones. In a female mammal, the F factor is calculated by dividing the product of the serum concentration of estrogen times the serum concentration of sex hormone-binding globulin times a conversion factor, by the fasting serum concentration of insulin. In a male mammal, the F factor is calculated by dividing the product of the serum concentration of testosterone times a conversion factor, by the product of the serum concentration of sex hormone-binding globulin times the fasting serum concentration of insulin. The F factor is then compared to a prescription plan to determine if the mammal has a hormone disorder or is at risk of developing a hormone disorder. From this prescription plan a dose of a therapeutic agent is determined to achieve a therapeutic F factor in the mammal having been identified as having a hormone disorder, or at risk of developing a hormone disorder. The therapeutic agent is then administered at the determined dose, and the method is repeated until a therapeutic F factor is achieved in the mammal. The present invention is also directed to method, kits, combinations, and compositions for treating, preventing or reducing the risk of developing a hormone disorder, or a disease or disorder associated with, or related to, a hormone disorder, such as cardiovascular disease.

### BRIEF DESCRIPTION OF THE DRAWINGS

FIG. 1 is a graph illustrating serum glucose levels versus administration of testosterone over time and amount of insulin administered versus testosterone injection over time in a woman.

FIG. 2 is a graph illustrating serum glucose levels versus testosterone injection over time and Micronase (sulfylurea/glyburide) levels versus testosterone injections over time in a man.

FIG. 3 is a graph illustrating serum glucose levels versus testosterone administration over time and insulin use versus testosterone administration over time in an insulin resistant man.

FIG. 4 is a graph illustrating glycosylated hemoglobin (HbA,,) versus testosterone administration over time in a man.

FIG. 5 is a graph illustrating the differential effect of sex hormone-binding globulin (SHBG) on the percentage of tracer-treated testosterone and estradiol.

FIG. 6 is a graph illustrating the mean sex hormone-binding globulin versus insulin for current estrogen users.

FIG. 7 is a graph illustrating the mean sex hormone-binding globulin versus duration of time on estrogen replacement therapy (D-estrogen replacement therapy).

FIG. 8 is a graph illustrating the mean fasting insulin versus duration of time on estrogen replacement therapy (D-estrogen replacement therapy).

FIG. 9 is a graph illustrating the Free Insulin Testosterone Factor in men.

FIG. 10 is a graph illustrating the Free Insulin Testosterone Index in men and women.

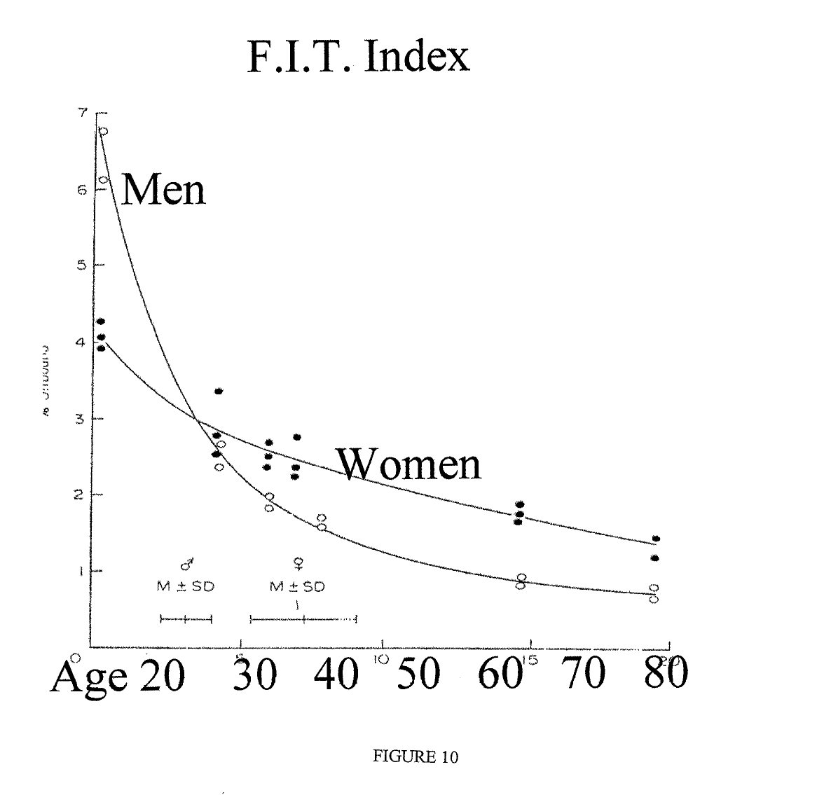

### DETAILED DESCRIPTION OF THE INVENTION

While the present invention may be embodied in many different forms, several specific embodiments are discussed herein with the understanding that the present disclosure is to be considered only as an exemplification of the principles of the invention, and it is not intended to limit the invention to the embodiments illustrated.

Where the invention is illustrated herein with particular reference to insulin, it is understood that any insulin produced naturally by the mammal, for example, by the beta cells in the islets of Langerhans and/or by recombinant DNA techniques and/or by semisynthetic processes, can, if desired, be substituted in whole or in part for insulin in the methods, kits, combinations, and compositions herein described. Where the invention is illustrated herein with particular reference to estrogen, it will be understood that any other estrogenic hormone can, if desired, be substituted in whole or in part for estrogen in the methods, kits, combinations, and compositions herein described. Where the invention is illustrated herein with particular reference to testosterone, it will be understood that any other steroid in the testosterone synthetic pathway can, if desired, be substituted in whole or in part for testosterone in the methods, kits, combinations, and compositions herein described. Where the invention is illustrated herein with particular reference to methyltestosterone, it will be understood that any other inhibitor of the synthesis of sex hormone-binding globulin can, if desired, be substituted in whole or in part for methyltestosterone in the methods, kits, combinations, and compositions herein described.

One embodiment of the present invention is directed to a method of identifying a mammal with a hormone disorder, or identifying a mammal at risk of developing a hormone disorder. The method comprises determining the serum concentration of estrogen, testosterone, sex hormone-binding globulin, and insulin in the mammal, and calculating their ratio, herein referred to as the “Free Insulin Testosterone” test, or the “F factor.” In a female mammal, the F factor is calculated by dividing the product of the serum concentration of estrogen times the serum concentration of sex hormone-binding globulin times a conversion factor, by the fasting serum concentration of insulin. In a male mammal, the F factor is calculated by dividing the product of the serum concentration of testosterone times a conversion factor, by the product of the serum concentration of sex hormone-binding globulin times the fasting serum concentration of insulin. A therapeutic agent is then administered to the mammal until a therapeutic F factor is achieved in the mammal. In one embodiment of the present invention, the therapeutic agent is administered once. In another embodiment of the present invention, the therapeutic agent is administered multiple times.

In one embodiment of the present invention, the F factor is compared to a graph plotting the Free Insulin Testosterone Index in a male and/or female subject, for example see Figure No. 10, to determine if the mammal has a hormone disorder or is at risk of developing a hormone disorder. From this graph a dose of a therapeutic agent is determined to achieve a therapeutic F factor in the mammal having been identified as having a hormone disorder, or at risk of developing a hormone disorder. The therapeutic agent is then administered at the determined dose, and the method is repeated until a therapeutic F factor is achieved in the mammal.

In a mammal, a therapeutic F factor is associated with a serum concentration ratio of insulin, estrogen, testosterone, and sex hormone-binding globulin that does not indicate that the mammal has a hormone disorder, or is at risk of developing a hormone disorder. In one embodiment of the present invention, a prescription plan is used to determine the dose of a therapeutic agent to be administered to the mammal to achieve a therapeutic F factor. The administration of a therapeutic agent to achieve a therapeutic F factor is thus useful in treating, preventing, or reducing the risk of developing a hormone disorder, or a disease or disorder associated with, or related to, a hormone disorder, such as cardiovascular disease. Achieving a therapeutic F factor in a mammal determined to have a non-therapeutic F factor before administering the therapeutic agents of the present invention is associated with treating, preventing or reducing the onset of cardiovascular disease, Alzheimer's disease, dementia, and cataracts; normalizing hypogonadism; improving sexual dysfunction; increasing libido; normalizing cholesterol levels; normalizing abnormal electrocardiograms of patients and improving vasomotor symptoms; improving diabetic retinopathy as well as lowering the insulin requirements of diabetic patients; decreasing the percentage of body fat; normalizing glucose levels; decreasing the risk factors for cardiovascular disease, including normalizing hypertension, and treating obesity; preventing osteoporosis, osteopenia, vaginal dryness, and thinning of the vaginal wall; relieving menopausal symptoms and hot flashes; improving cognitive dysfunction; and treating, preventing or reducing the risk of cancer, for example, cervical, uterine or breast cancer.

Therapeutic agents useful in the present invention, include, but are not limited to: estrogen; testosterone; sex hormone-binding globulin; insulin; a pharmaceutical agent that increases the serum concentration of estrogen, testosterone, sex hormone-binding globulin and/or insulin in a mammal; and a pharmaceutical agent that decreases the serum concentration of estrogen, testosterone, sex hormone-disorder globulin, and/or insulin in a mammal; and/or all salts, esters, amides, enantiomers, isomers, tautomers, prodrugs and derivatives of any of these compounds or agents.

The present invention also is directed to methods, kits, combinations, and compositions for treating, preventing or reducing the risk of developing a hormone disorder, or a disease or disorder associated with, or related to, a hormone disorder; or the symptoms associated with, or related to the hormone disorder, or the disease or disorder associated with, or related to, a hormone disorder, in a mammal in need thereof. The method comprises receiving a blood sample from a mammal and measuring the concentration of estrogen, testosterone, sex hormone-binding globulin, and insulin in the serum of the mammal. Once the concentration of the hormones is determined, an F factor is determined. In one embodiment of the present invention, the F factor is then compared to a prescription plan to determine the dose of a therapeutic agent to achieve a therapeutic F factor in the serum of the mammal.

The present invention includes methods, kits, combinations, and compositions for reversing, halting or slowing the progression of a hormone disorder once it becomes clinically evident, and/or halting or slowing the progression of a disease or disorder associated with, or related to, a hormone disorder once it becomes clinically evident, and/or treating the symptoms associated with, or related to a hormone disorder, and/or treating the symptoms associated with, or related to the disease or disorder associated with, or related to, a hormone disorder. The mammal can already have a hormone disorder, or a disease or disorder associated with, or related to, a hormone disorder at the time of administration, or be at risk of developing a hormone disorder, or a disease or disorder associated with, or related to, a hormone disorder.

The present invention also includes kits comprising a hormone disorder-effective amount of at least one of estrogen; testosterone; sex hormone-binding globulin; insulin; a pharmaceutical agent that increases the serum concentration of estrogen, testosterone, sex hormone-binding globulin and/or insulin in a mammal; and a pharmaceutical agent that decreases the serum concentration of estrogen, testosterone, sex hormone-disorder globulin, and/or insulin in a mammal; and/or all salts, esters, amides, enantiomers, isomers, tautomers, prodrugs and derivatives of these compounds or agents.

The kit can also contain a set of instructions for the patient.

Besides being useful for both male or female human treatment, the present invention is also useful for veterinary treatment of companion mammals, exotic animals and farm animals, including mammals, rodents, and the like. In one embodiment, the mammals include horses, dogs, and cats.

In one embodiment of the present invention, the serum concentration of the hormone estrogen, testosterone, sex hormone-binding globulin, and insulin are measured in a mammal, and a “F factor” is calculated. The “F factor” in a female mammal is determined by the following equation:

F_{f}=(E×S×Q)/N

The “F factor” in a male mammal is determined by the following equation:

Fₘ=(T×P)/(S×N)

In both equations, E is estrogen serum concentration in mg/dl; S is sex hormone-binding globulin serum concentration in nmol/L; Q is a conversion factor; N is fasting insulin serum concentration in mIU/ml; T is testosterone serum concentration in ng/dl; and P is a conversion factor.

In one embodiment of the present invention, the conversion factor Q is equal to a factor that provides a therapeutic F factor equal to, or greater than, 100 for a young healthy eighteen-year-old female.

For example, in a young healthy menstruating female, serum concentrations of estrogen generally peak on day 23 of the menstrual cycle. The estrogen values range, for example, from about 50 mg/dl to about 250 mg/dl, or higher depending on the day of the cycle. However, this is not a factor when considering risk factors in post-menopausal women. Normal serum testosterone levels for females range from about 30 ng/dl to about 100 ng/dl, and normal serum concentrations of sex hormone-binding globulin are generally greater than 40 nmol/L. Therefore, a healthy eighteen-year-old having an estrogen serum concentration of 250 mg/dl, a serum concentration of sex hormone-binding globulin of 100 nmol/L, a fasting serum insulin concentration of 5 mIU/ml, and Q is 0.02, has an F factor of 100.

Once a conversion factor has been determined for a healthy young mammal, the conversion factor is then used to determine the F factor for other subjects. For example, in a fifty-year-old female, having an estrogen serum concentration of about 30 mg/dl to about 50 mg/dl, a serum concentration of sex hormone-binding globulin of about 20 nmol/L to about 50 nmol/L, and a fasting serum insulin concentration of about 10 mIU/ml to about 15 mIU/ml, has an F factor of 8 to 12, and from 16 to 50, when Q is 0.02. In a sixty-year-old diabetic female, for example, having an estrogen serum concentration of about 30 mg/dl to about 50 mg/dl, a serum concentration of sex hormone-binding globulin of about 20 nmol/L to about 50 nmol/L, and a fasting serum insulin concentration of about 40 mIU/ml, has an F factor of 3 to 5, when Q is 0.02.

In another embodiment of the present invention, the conversion factor P is equal to a factor that provides a therapeutic F factor equal to, or greater than, 100 for a young healthy eighteen-year-old male.

For example, in a healthy male, serum concentrations of testosterone range from about 300 ng/dl to about 1200 ng/dl, and serum concentration of sex hormone-binding globulin are generally less than 75 nmol/L.

In an eighteen-year-old male, for example, having a testosterone serum concentration of 600 ng/dl, a serum concentration of sex hormone-binding globulin of 10 nmol/l, and a fasting serum insulin concentration of 6 mIU/ML, has an F factor equal to 100, when P is equal to the product of 0.5 nmol/L times the fasting insulin serum concentration in mIU/ml.

In a fifty-year-old fairly healthy male, for example, a testosterone serum concentration of about 350 ng/dl to about 500 ng/dl, a serum concentration of sex hormone-binding globulin of about 10 to about 25nmol/l, and a fasting serum insulin concentration of about 7mIU/ml to about 10 mIU/ml, provides an F factor of about 50 to about 90 and about 14 to about 20, when P is equal to the product of 0.5 nmol/L times the fasting insulin serum concentration in mIU/ml.

A fifty-year-old insulin dependent diabetic male, for example, having a testosterone serum concentration of 200 ng/dl, a serum concentration of sex hormone-binding globulin of 60 nmol/l, and fasting serum insulin concentration (or insulin requirements) of 40 mIU/ml, has an F factor of less than 1, when P is equal to the product of 0.5 nmol/L times the fasting insulin serum concentration in mIU/ml.

As used herein, the term “therapeutic F factor” refers to an F factor associated with little or no occurrence of a hormone disorder in a mammal, or a disease associated with, or related to, a hormone disorder in a mammal; or a low or moderate risk of a mammal developing a hormone disorder, or a disease associated with, or related to, a hormone disorder. A “non therapeutic F factor” is associated with, or related to, a mammal having a hormone disorder, or a disease associated with, or related to, a hormone disorder, or at risk of developing a hormone disorder, or a disease associated with, or related to a hormone disorder. As a way of illustration, the examples herein use a therapeutic F factor equal to, or greater than, 100 while a non-therapeutic F factor is less than 100. The number that is selected for the therapeutic F factor is not critical, as it is used as a means of using young healthy disease-free mammals to identify and treat mammals that have a hormone disorder, or at risk of developing a hormone disorder, or have a disease or disorder associated with, or related to, a hormone disorder.

It is understood, however, that specific conversion factors and/or F factors that may be used in the present invention for any particular patient depends upon a variety of factors including, but not limited to, the age, body weight, general health, sex, and diet of the patient.

In one embodiment, the dosage of the therapeutic agent of the present invention for a man or a women is determined by reference to FIG. No. 10, in which the Free Index Testosterone Index moves from the right side of the graph toward the maximum noted by the left side of the curve, that is, a Free Index Testosterone Index of a healthy twenty-year-old subject.

In one embodiment of the present invention, the F factor is used to monitor the hormone balance in a mammal once treatment begins, and also to monitor the most effective dosage of therapeutic agents during treatment of a hormone disorder, or a symptom associated with, or related to, a hormone disorder in a mammal. For example, in a male mammal with low testosterone serum concentrations and high levels of sex hormone-binding globulin, testosterone is administered over a period of time to deliver a subsequent fall in insulin serum concentration and achieving a therapeutic F factor. During treatment to achieve a therapeutic F factor, the serum hormone concentration of the mammal is periodically monitored, and by reference to the prescription plan, the dose of testosterone is adjusted to achieve or maintain a therapeutic F factor.

While not wishing to be bound by theory, it is believed that insulin and oral hypoglycemic agents derive their hypoglycemic effects by increasing utilization of exogenous or endogenous insulin, and a pharmacological dose of testosterone administered to a mammal lowers tissue glucose/glycogen levels and corrects the physiological basis of insulin resistance. Following administration of testosterone, it is contemplated that both men and women patients will experience lowering of serum glucose, hyperglycemia, and/or lowering of insulin levels, hyperinsulinemia. Both clinically and under biological test conditions, both men and women confirm a lowering of insulin resistance as noted with a decrease in hemoglobin AIC. It is believed that the medical basis for the normalization of serum glucose in both insulin and non-insulin dependent adult-onset diabetes is thought to be that testosterone changes insulin resistance at the cellular level. By reversing tissue resistance to the movement of glucose into the muscle and liver cell, testosterone increases the tissue's sensitivity to insulin. The result is that the level of glucose in the serum decreases and the amount of insulin needed to effect this drop in serum glucose is reduced. It is also contemplated that supraphysiologic levels of testosterone slow the conversion of glycogen into glucose.

It is also believed that the mode of action of testosterone is manyfold. For example, and not wishing to be bound by theory, it is believed that testosterone utilizes intracellular glycogen. Previously reported tissue biopsies confirm that the diabetic individual has elevated glycogen stores within the muscle cell, and that testosterone significantly lowers the amount of glycogen within the muscle cell. Thus, it is believed that testosterone increases cellular utilization of intracellular glycogen. Additionally, it is believed that testosterone by its action on sex hormone-binding globulin lowers insulin resistance. Insulin does not change sex hormone-binding globulin; rather, a change in sex hormone-binding globulin changes insulin release. By way of example, it is contemplated that the utilization of various forms of testosterone results in a drop in serum glucose. Thus, the amount of insulin needed to stabilize a given glucose load will be reduced. Therefore, testosterone lowers serum blood levels and lowers insulin resistance. Testosterone, by the actions of utilization of intracellular glycogen and its action on sex hormone-binding globulin, allows the liver cells to reduce the storage of glycogen from the excess blood glucose.

It is also believed that testosterone lowers normal fructosamine levels by the fact that the liver cells are able to stop storing excess glucose as intracellular glycogen could produce liver cells that are able to produce additional insulin-like growth factor-1]. Therefore, testosterone, by clearing intracellular glycogen, raises liver production of insulin-like growth factor-1.

It is also believed that the utilization of injectable and subcutaneous pellets of testosterone lowers serum concentrations of sex hormone-binding globulin. However, depending on the diabetic state, there is a physiologic limit to the decrease in sex hormone-binding globulin. Once this maximum individual effect is realized, higher physiologic levels of testosterone offer no additional benefit. Subcutaneous and intramuscular androgens have similar effects on sex hormone-binding globulin and, therefore, insulin resistance in tissue. However, oral androgens, testosterone creams, gels and transdermal delivery systems do not have this same positive effect on insulin resistance and/or sex hormone-binding globulin. Testosterone increases diabetic male erectile performance. This may be related to an increase in total testosterone, free or unbound testosterone, an improvement in circulation or a decrease in sex hormone-binding globulin.

Without wishing to be bound by theory, it is also believed that changes in serum sex hormone-binding globulin concentration produce a much greater alteration of percentage unbound testosterone than estradiol. This is partly because estradiol binds less well to sex hormone-binding globulin than testosterone, and partly because estradiol binds better to albumin than testosterone. It is also believed that increasing testosterone concentration over the physiological range does not increase the unbound estradiol; and similarly a marked increase in cortisol does not significantly increase the unbound testosterone by displacement of CBG. Additionally, it is also believed that a drop in sex hormone-binding globulin serum concentration decreases the availability of biologically active estradiol and would be detrimental for the female patient.

Also, it is believed that changes in sex hormone-binding globulin influence insulin resistance directly and thereafter serum levels of insulin.

In one embodiment of the present invention, the insulin that can be used in the methods, kits, combinations, and compositions of the present invention is naturally occurring insulin. Other insulin that may be used in partial or complete replacement of naturally occurring insulin is insulin produced by recombinant DNA techniques or by semisynthetic techniques; and all salts, esters, amides, enantiomers, isomers, tautomers, prodrugs and derivatives of these compounds. Insulin can also include the active site of insulin, or recombinant DNA that codes for the active site. Combinations of insulin can also be used in the present invention.

In another embodiment of the present invention, the estrogenic hormone that can be used in the methods, kits, combinations, and compositions of the present invention is the naturally occurring estrogen 17 beta-estradiol (beta-estradiol; 1, 3, 5(10)-estratriene-3, 17 beta-diol). Estrogen occurs in at least two isomeric forms, including beta estrogen and alpha estrogen. Beta estrogen is the beta isomer of estrogen compounds. Alpha estrogen is the alpha isomer of estrogen components. The term “estradiol” is either alpha or beta estradiol unless specifically identified. Other estrogenic steroid hormones can be used in partial or complete replacement of 17 beta-estradiol, for example, an ester which is biologically compatible and can be absorbed effectively transdermally. The estradiol esters can be, illustratively, estradiol-3,17-diacetate; estradiol-3-acetate; estradiol-17-acetate; estradiol-3,17-divalerate; estradiol-3-valerate; estradiol-17-valerate 3-mono, 17-mono and 3,17-dipropionate esters, corresponding cypionate, heptanoate, benzoate and the like esters; ethynyl estradiol; estrone and other estrogenic steroids and salts, enantiomers, isomers, tautomers, prodrugs and derivatives thereof that are possible to administer by transdermal route. Other estrogen-related compounds that may be used in the methods, kits, combinations, and compositions of the present invention include, but are not limited to, conjugated estrogens (including estrone sulfate, equilin, and 17-alpha-dihydroequilin), estradiol valerate, estriol, estrone, estrone sulfate, estropipate, ethinyl estradiol, mestranol, and all salts, esters, amides, enantiomers, isomers, tautomers, prodrugs and derivatives of these compounds.

Estrogenic hormones are currently available in various formulations including, but not limited to, those available as a cream, pessary, vaginal ring, vaginal tablet, transdermal preparation, gel, and oral tablet. Examples of vaginal creams include PREMARIN® (conjugated estrogen), ORTHO DIENOSTEROL® (dienosterol), and OVESTIN® (estriol). Available pessary formulations include ORTHO-GYNEST® (estriol), and TAMPOVAGAN® (stilbestrol). An example of a vaginal ring formulation is ESTRING® (estradiol), and an example of a vaginal tablet is VAGIFEM® (estradiol). Available transdermal estrogen preparations containing estradiol include ERC ALORA®, CLIMARA®, DERMESTRIL®, ESTRADERM®, ESTRADERM® TTS, ESTRADERM® MX, EVOREL®, FEMATRIX®, FEMPATCH®, FEMSEVEN®, MENOREST(®, PROGYNOVA® TS, and VIVELLE®. Available estrogen gels containing estradiol include ESTROGEL® and SANDRENA®. Estradiol is also available formulated as an implant pellet, for example, ESTRADIOL IMPLANT®. Tablet formulations include PREMARIN® (conjugated estrogen), ESTRATAB® (esterified estrogen), ESTRATEST® (esterified estrogen, methyltestosterone), MENEST® (esterified estrogen), CLIMAGEST®, (estradiol), CLIMAVAL® (estradiol), ELLESTE SOLO® (estradiol), ESTRACE® (estradiol), PROGYNOVA® (estradiol), ZUMENON® (estradiol), HORMONIN® (estradiol, estrone, estriol), HARMOEN® (estrone), OGEN® (estropipate), and ORTHO-EST® (estropipate).

Included in the definition of “estrogenic hormones” are non-steroidal estrogens known to those skilled in the art. Other estrogen compounds included in the definition of “estrogenic hormones” are estrogen derivatives, estrogen metabolites and estrogen precursors as well as those molecules capable of binding cell associated estrogen receptor as well as other molecules where the result of binding specifically triggers a characterized estrogen effect.

Combinations of the above-mentioned estrogenic hormones can also be used in the present invention.

A class of steroids in the testosterone synthetic pathway useful in the methods, kits, combinations, and compositions of the present invention include steroids in the testosterone synthetic pathway, including steroids in the testosterone anabolic or catabolic pathway. In a broad aspect of the invention, the active ingredients employed in the composition may include anabolic steroids such as androisoxazole, bolasterone, clostebol, ethylestrenol, formyldienolone, 4-hydroxy-19-nortestosterone, methenolone, methyltrienolone, nandrolone, oxymesterone, quinbolone, stenbolone, trenbolone; androgenic steroids such as boldenone, fluoxymesterone, mestanolone, mesterolone, methandrostenolone, 17-methyltestosterone, 17 alpha-methyl-testosterone 3-cyclopentyl enol ether, norethandrolone, normethandrone, oxandrolone, oxymetholone, prasterone, stanlolone, stanozolol, dihydrotestosterone, testosterone; and progestogens such as anagestone, chlormadinone acetate, delmadinone acetate, demegestone, dimethisterone, dihydrogesterone, ethinylestrenol, ethisterone, ethynodiol, ethynodiol diacetate, flurogestone acetate, gestodene, gestonorone caproate, haloprogesterone, 17-hydroxy-16-methylene-progesterone, 17 alpha-hydroxyprogesterone, 17 alpha-hydroxyprogesterone caproate, medrogestone, medroxyprogesterone, megestrol acetate, melengestrol, norethindrone, norethindrone acetate, norethynodrel, norgesterone, norgestimate, norgestrel, norgestrienone, 19-norprogesterone, norvinisterone, pentagestrone, progesterone, promegestone, quingestrone, and trengestone; and all salts, esters, amides, enantiomers, isomers, tautomers, prodrugs and derivatives of these compounds (based upon the list provided in The Merck Index, Merck & Co. Rahway, N.J.(1998)). Combinations of the above-mentioned steroids can also be used in the present invention.

Dosages of testosterone can be given in such a manner to maintain serum levels at twice the considered maximum range for a mammal. For example, if a standard reference laboratory has a normal range of serum testosterone of 50-1200 mg/dl, then by the present methodology, the therapeutic range of testosterone dosing would be reached with either frequent injections of testosterone or by placing testosterone pellets in the patient until the serum level approximated 2000-2800 mg/dl.

In one embodiment, testosterone is administered to a mammal at dosage levels in the range of about 200 to about 1200 mg of parenteral testosterone per month. For example, for a normal human adult having a body weight of about 70 kilograms, a dosage in the range of about 15 mg to about 40 mg of testosterone per kilogram of body weight per day is administered. The specific dosage used, however, can vary. For example, the dosage can depend on a number of factors including the requirements of the patient, the severity of the condition being treated, and the pharmacological activity of tile compound being used. For example, transdermal dosage is 50-100% higher due to absorption differences from transdermal to intramuscular or subcutaneous placement. With the addition of the Free Insulin Testosterone test, the determination of optimum dosages for a particular patient is well known to those skilled in the art.

In one embodiment, a hormone disorder-effective amount of testosterone can be achieved by placing about 200 mg to about 900 mg of testosterone pellets subcutaneously in the abdominal fat or hip fat pads every one to two months. The total dosage of testosterone pellets and the frequency may be increased until the testosterone therapeutic range is reached. There are clinical effects of testosterone on insulin resistance, including, but not limited to, the sex hormone-binding globulin concentration is reduced or stabilized, and/or the F factor is in a therapeutic range. Testosterone also does not induce drug reactions or side effects when combined with either insulin or hypoglycemic agents, and testosterone does not induce insulin coma or hypoglycemia at a physiologically acceptable dosage.

In one embodiment of the present invention, testosterone is administered to a mammal in cases of insulin resistance.

In another embodiment of the present invention, testosterone is administered to a mammal to enhance the action of insulin and/or hypoglycemic agents.

In another embodiment of the present invention, testosterone is administered to a mammal to lower serum cholesterol, trigylcerides and/or to increase high-density lipoproteins.

A class of pharmaceutical agents that increases sex hormone-binding globulin levels in the serum of a mammal useful in the methods, kits, combinations, and compositions of the present invention includes pharmaceutical agents that stimulate the synthesis of the sex hormone-binding globulin. A specific compound of interest that stimulates the synthesis the sex hormone-binding globulin includes, but is not limited to, estrogen, and all salts, esters, amides, enantiomers, isomers, tautomers, prodrugs and derivatives thereof. Combinations of pharmaceutical agents that increase serum levels of sex hormone-binding globulin can also be used in the present invention. Additionally, pregnancy dramatically raises sex hormone-binding globulin serum concentration levels as a result of the production of large amounts of placental estrogens.

A class of pharmaceutical agents, herein termed “insulin inhibiting agents,” which decrease serum concentration of insulin in a mammal useful in the methods, kits, combinations, and compositions of the present invention, includes pharmaceutical agents or “insulin inhibiting agents” that decrease the serum concentration or activity of insulin or inactivate or otherwise antagonize or inhibit the activity, synthesis, or metabolism of insulin. The insulin inhibiting agents include antagonists and agonists of insulin that counteract the action of insulin. Without wishing to be bound by theory, insulin inhibiting agents used in the present invention may act, for example, by binding to the insulin receptor, interfering with nuclear accumulation of active receptor-hormone complexes, by down-regulating the synthesis insulin or acting on the metabolism of insulin. Specific insulin inhibiting agents of interest that may be used in the present invention include, but are not limited to, testosterone in men and estrogen in women; and all salts, esters, amides, enantiomers, isomers, tautomers, prodrugs and derivatives of these insulin inhibiting agents. Combinations of insulin inhibiting agents can also be used in the present invention.

A class of pharmaceutical agents, herein termed “estrogen inhibiting agents,” which decrease serum concentration of estrogenic hormones, for example, estradiol, in a mammal useful in the methods, kits, combinations, and compositions of the present invention, includes pharmaceutical agents or “estrogen inhibiting agents” that decrease the serum concentration or activity of estrogen or inactivate or otherwise antagonize or inhibit the activity, synthesis, or metabolism of estrogen. The estrogen inhibiting agents include antagonists and agonists of estrogen that counteract the action of estrogen. Without wishing to be limited to scientific theories, estrogen inhibiting agents used in the present invention may act, for example, by binding to the androgen receptor, interfering with nuclear accumulation of active receptor-hormone complexes, by down-regulating the synthesis estrogen or acting on the metabolism of estrogen. Specific estrogen inhibiting agents of interest that may be used in the present invention include, but are not limited to, (1) steroidal anti-androgens, for example, cytoproterone acetate, and megestrol acetate; (2) pure anti-androgens, for example, flutamide (DROGENIL1™), nilutamide, and bicalutamide (CADOLEX™); (3) androgen synthesis inhibitors (for example, ketoconazole); (4) 5-alpha-reductase inhibitors, for example, finasteride (PROPECIA™); (5) androgen receptor antagonists including cytoproterone, flutamide, cymetidine, ranitidine and spironolactone; and (6) agonists and antagonists of the luteinizing hormone releasing hormone, and all salts, esters, amides, enantiomers, isomers, tautomers, prodrugs and derivatives of these estrogen inhibiting agents. Combinations of estrogenic inhibiting agents can also be used in the present invention.

A class of pharmaceutical agents, herein termed “testosterone inhibiting agents,” which decrease serum concentration of testosterone in a mammal useful in the methods, kits, combinations, and compositions of the present invention, includes pharmaceutical agents or “testosterone inhibiting agents” that decrease the serum concentration or activity of testosterone or inactivate or otherwise antagonize or inhibit the activity, synthesis, or metabolism of testosterone. The testosterone inhibiting agents include antagonists and agonists of testosterone that counteract the action of testosterone. Without wishing to be limited to scientific theories, testosterone inhibiting agents used in the present invention may act, for example, by binding to the androgen receptor, interfering with nuclear accumulation of active receptor-hormone complexes, by down-regulating the synthesis estrogen or acting on the metabolism of testosterone. The testosterone-inhibiting agents that may be used in the present invention include, but are not limited to: (1) hormones which inhibit hypothalamic release of gonadotrophin releasing hormone; (2) gonadotrophin releasing hormone analogues (for example, goserelin ZOLADEX™), leuprorelin (PROSTAP™), buserelin (SUPRELFACT™), triptorelin (De-capeptyl), (nafarelin); (3) steroidal anti-androgens (for example, cytoproterone acetate, megestrol acetate); (4) pure anti-androgens (for example, flutamide (DROGENIL™), nilutamide, bicalutamide (CADODEX™); (5) androgen synthesis inhibitors (for example, ketoconazole); (6) 5-alpha-reductase inhibitors, for example, finasteride (PROPECIA™); (7) androgen receptor antagonists including cytoproterone, flutamide, cymetidine, ranitidine and spironolactone; (8) agonists and antagonists of the luteinizing hormone releasing hormone; and (9) aromatase inhibitors, for example, anastrozole, fadrozole, letrozole, vorozole, roglethimide, atamestane, exemestane, formestane, and all salts, esters, amides, enantiomers, isomers, tautomers, prodrugs and derivatives of these testosterone inhibiting agents. Combinations of testosterone inhibiting agents can also be used in the present invention.

Also included in the methods, kits, combinations, and compositions of the present invention are peptide analogs of luteinizing hormone releasing hormone, and all salts, esters, amides, enantiomers, isomers, tautomers, prodrugs and derivatives of these testosterone inhibiting agents. Combinations of peptide analogs of luteinizing hormone releasing hormone can also be used in the present invention. While not wishing to be bound by theory, it is believed that the peptide analogs of luteinizing hormone releasing hormone are agonists of luteinizing hormone releasing hormone receptors and, if administered to a mammal, suppress the production and release of luteinizing hormone releasing hormone in the hypophysis to reduce the responsiveness of the testes and ovaries to luteinizing hormone releasing hormone and consequently reduce the secretion of testosterone and estrogen and lowering testosterone and estrogen serum concentration levels.

A class of pharmaceutical agents that decrease the sex hormone-binding globulin levels in a mammal useful in the methods, kits, combinations, and compositions of the present invention includes pharmaceutical agents that decrease the serum concentration or activity of sex hormone-binding globulin or inactivate or otherwise antagonize or inhibit the activity, synthesis, or metabolism of sex hormone-binding globulin. Sex hormone-binding globulin is a serum protein, and is known to bind to testosterone and estrogen, effecting the biological activity of these hormones. Specific compounds of interest include, but are not limited to, methyltestosterone and fluoxymesterone, and all salts, esters, amides, enantiomers, isomers, tautomers, prodrugs and derivatives of these compounds, and estrogen blockers, including, but not limited to, Arimidex™. Methyltestosterone is currently available in various formulations including those available orally, for example ANDROID® and TESTRED®. Fluoxymesterone is also currently available in various formulations including those available orally, for example, HALOSTESTIN®. Combinations of pharmaceutical agents that decrease serum levels of sex hormone-binding globulin can also be used in the present invention.

While not wishing to be bound by theory, is it believed that the sex hormone-binding globulin serves as the important determinant of the ratio of unbound estrogen and unbound testosterone. In female systems, increases in sex hormone-binding globulin have a feminizing effect while decreases in sex hormone-binding globulin are masculinizing. In addition to being directly effected by the level of various sex hormones, sex hormone-binding globulin is also strongly influenced by states of hyperinsulinemia and obesity. Hyperinsulinemia with and without obesity has been shown to be masculinizing for the polycystic female.

In one embodiment, the present invention is directed to a method of monitoring hormone levels in a mammal. The method comprises receiving a blood sample from a mammal; measuring the serum concentration of insulin, estrogen, testosterone, and sex hormone-binding globulin in the blood sample, and determining an F factor.

In one embodiment of the present invention, the F factor for a female mammal is F=(E×S×Q)/N, the F factor for a male mammal is F=(T×P)/(S×N), and wherein E is estrogen serum concentration, S is sex hormone-binding globulin serum concentration, Q is a conversion factor, N is fasting insulin serum concentration, T is testosterone serum concentration, and P is a conversion factor. In another embodiment, the conversion factor Q is 0.02, and the conversion factor P is equal to the product of 0.5 nmol/L times the fasting insulin serum concentration.

In another embodiment, the present invention is directed to a method for treating, preventing, or reducing the risk of developing a hormone disorder in a male or female mammal in need thereof. The method comprises measuring the serum concentration of insulin, estrogen, testosterone, and sex hormone-binding globulin in the mammal; determining an F factor; receiving a prescription plan; calculating from the prescription plan based on the F factor a dose of a therapeutic agent, wherein the therapeutic agent comprises at least one of insulin, an estrogenic hormone, a steroid in the testosterone synthetic pathway, a sex hormone-binding globulin, an insulin inhibiting agent, an estrogen inhibiting agent, a testosterone inhibiting agent, a sex hormone-binding globulin inhibiting agent, a pharmaceutical agent that increases the serum concentration of estrogen, a pharmaceutical agent that increases the serum concentration of testosterone, a pharmaceutical agent that increases the serum concentration of sex hormone-binding globulin, and a pharmaceutical agent that increases the serum concentration of insulin in the mammal, administering to the mammal the therapeutic agent, and if necessary repeating the method until a desired amount of the therapeutic agent is administered to the mammal to provide a therapeutic F factor.

In another embodiment, the present invention is directed to a method for treating, preventing, or reducing the risk of developing a disorder associated with, or related to, a hormone disorder in a male or female mammal in need thereof. The method comprises measuring the serum concentration of insulin, estrogen, testosterone, and sex hormone-binding globulin in the mammal, determining an F factor, receiving a prescription plan, calculating from the prescription plan based on the F factor a dose of a therapeutic agent, wherein the therapeutic agent comprises at least one of insulin, an estrogenic hormone, a steroid in the testosterone synthetic pathway, a sex hormone-binding globulin, an insulin inhibiting agent, an estrogen inhibiting agent, a testosterone inhibiting agent, a sex hormone-binding globulin inhibiting agent, a pharmaceutical agent that increases the serum concentration of estrogen, a pharmaceutical agent that increases the serum concentration of testosterone, a pharmaceutical agent that increases the serum concentration of sex hormone-binding globulin, and a pharmaceutical agent that increases the serum concentration of insulin in the mammal, administering to the mammal the therapeutic agent, and if necessary repeating the method until a desired amount of the therapeutic agent is administered to the mammal to provide a therapeutic F factor.

In another embodiment of the present invention, the disorder associated with, or related to, a hormone disorder is cardiovascular disease.

In yet another embodiment, the present invention is directed to a method for restoring the hormone balance of a mammal in need thereof. The method comprises receiving a blood sample from the mammal, measuring the serum concentration of insulin, estrogen, testosterone, and sex hormone-binding globulin in the mammal, determining an F factor; receiving a prescription plan, calculating a dose of a therapeutic agent from the prescription plan based on the F factor, wherein the therapeutic agent comprises at least one of insulin, an estrogenic hormone, a steroid in the testosterone synthetic pathway, a sex hormone-binding globulin, an insulin inhibiting agent, an estrogen inhibiting agent, a testosterone inhibiting agent, a sex hormone-binding globulin inhibiting agent, a pharmaceutical agent that increases the serum concentration of estrogen, a pharmaceutical agent that increases the serum concentration of testosterone, a pharmaceutical agent that increases the serum concentration of sex hormone-binding globulin, and a pharmaceutical agent that increases the serum concentration of insulin in the mammal, administering to the mammal the therapeutic agent at the calculated dose, and repeating the method until the hormone ratio of the mammal is about, or equal to, that of a young healthy disease-free mammal, that is, until a therapeutic F factor is achieved and/or maintained in the treated mammal.

In yet another embodiment, the present invention is directed to a kit for monitoring the hormone balance in a mammal and/or treating a hormone disorder, and/or a disorder associated with, and/or related to, a hormone disorder.

In one embodiment, the present invention is directed to a method of decreasing insulin serum concentration levels in a mammal suffering from, and/or at risk of developing, a hormone disorder, or a disease or disorder associated with, or related to, a hormone disorder.

In another embodiment, the present invention is directed to a method of decreasing free and total estrogen serum concentration levels in a mammal suffering from, and/or at risk of developing, a hormone disorder, or a disease or disorder associated with, or related to, a hormone disorder.

In yet another embodiment, the present invention is directed to a method of decreasing free and total testosterone serum concentration levels in a mammal suffering from, and/or at risk of developing, a hormone disorder, or a disease or disorder associated with, or related to, a hormone disorder.

In still another embodiment, the present invention is directed to a method of decreasing sex hormone-binding globulin levels in the serum in a mammal suffering from, and/or at risk of developing, a hormone disorder, or a disease or disorder associated with, or related to, a hormone disorder.

In one embodiment of the present invention, a pharmaceutical agent for increasing insulin serum concentration levels is administered to a mammal suffering from, and/or at risk of developing, a hormone disorder, or a disease or disorder associated with, or related to, a hormone disorder.

In another embodiment of the present invention, a pharmaceutical agent for increasing free and total estrogen serum concentration levels is administered to a mammal suffering from, and/or at risk of developing, a hormone disorder, or a disease or disorder associated with, or related to, a hormone disorder.

In yet another embodiment of the present invention, a pharmaceutical agent for increasing free and total testosterone serum concentration levels is administered to a mammal suffering from, and/or at risk of developing, a hormone disorder, or a disease or disorder associated with, or related to, a hormone disorder.

In another embodiment, the present invention is directed to a method of increasing sex hormone-binding globulin serum concentration levels in a mammal suffering from, and/or at risk of developing, a hormone disorder, or a disease or disorder associated with, or related to, a hormone disorder.

In one embodiment, the present invention is directed to a method of increasing free and total estrogen serum concentration levels in a female mammal suffering from, and/or at risk of developing, a hormone disorder, or a disease or disorder associated with, or related to, a hormone disorder.

In yet another embodiment, the present invention is directed to a method of increasing free and total testosterone serum concentration levels in a female mammal suffering from, and/or at risk of developing, a hormone disorder, or a disease or disorder associated with, or related to, a hormone disorder.

In still another embodiment, the present invention is directed to a method of increasing sex hormone-binding globulin levels in the serum in a female mammal suffering from, and/or at risk of developing, a hormone disorder, or a disease or disorder associated with, or related to, a hormone disorder.

In one embodiment, the present invention is directed to a method of decreasing free and total estrogen serum concentration levels in a female mammal suffering from, and/or at risk of developing, a hormone disorder, or a disease or disorder associated with, or related to, a hormone disorder.

In yet another embodiment, the present invention is directed to a method of decreasing free and total testosterone serum concentration levels in a female mammal suffering from, and/or at risk of developing, a hormone disorder, or a disease or disorder associated with, or related to, a hormone disorder.

In still another embodiment, the present invention is directed to a method of decreasing sex hormone-binding globulin levels in the serum in a female mammal suffering from, and/or at risk of developing, a hormone disorder, or a disease or disorder associated with, or related to, a hormone disorder.

In another embodiment, the present invention is directed to a method of increasing free and total estrogen serum concentration levels in a male mammal suffering from, and/or at risk of developing, a hormone disorder, or a disease or disorder associated with, or related to, a hormone disorder.

In yet another embodiment, the present invention is directed to a method of increasing free and total testosterone serum concentration levels in a male mammal suffering from, and/or at risk of developing, a hormone disorder, or a disease or disorder associated with, or related to, a hormone disorder.

In still another embodiment, the present invention is directed to a method of increasing sex hormone-binding globulin levels in the serum in a male mammal suffering from, and/or at risk of developing, a hormone disorder, or a disease or disorder associated with, or related to, a hormone disorder.

In another embodiment, the present invention is directed to a method of decreasing free and total estrogen serum concentration levels in a male mammal suffering from, and/or at risk of developing, a hormone disorder, or a disease or disorder associated with, or related to, a hormone disorder.

In yet another embodiment, the present invention is directed to a method of decreasing free and total testosterone serum concentration levels in a male mammal suffering from, and/or at risk of developing, a hormone disorder, or a disease or disorder associated with, or related to, a hormone disorder.

In still another embodiment, the present invention is directed to a method of decreasing sex hormone-binding globulin levels in the serum in a male mammal suffering from, and/or at risk of developing, a hormone disorder, or a disease or disorder associated with, or related to, a hormone disorder.

Also included in the methods, kits, combinations, and compositions of the present invention are pharmaceutical compositions comprising an insulin deficient-effective amount of naturally occurring, recombinant, and/or semisynthetic insulin.

Also included in the methods, kits, combinations, and compositions of the present invention are pharmaceutical compositions comprising an estrogen deficient-effective amount of estrogen.

Also included in the methods, kits, combinations, and compositions of the present invention are pharmaceutical compositions comprising a testosterone deficient-effective amount of testosterone.

Also included in the methods, kits, combinations, and compositions of the present invention are pharmaceutical compositions comprising a sex hormone-binding globulin deficient-effective amount of a pharmaceutical agent for increasing sex hormone-binding globulin serum concentration levels.

Also included in the methods, kits, combinations, and compositions of the present invention are pharmaceutical compositions comprising a hormone disorder-effective amount of a pharmaceutical agent for decreasing insulin serum concentration levels, such as an insulin inhibiting agent.

Also included in the methods, kits, combinations, and compositions of the present invention are pharmaceutical compositions comprising a hormone disorder-effective amount of a pharmaceutical agent for decreasing free and total estrogen serum concentration levels, such as a estrogen inhibiting agent.

Also included in the methods, kits, combinations, and compositions of the present invention are pharmaceutical compositions comprising a hormone disorder-effective amount of a pharmaceutical agent for decreasing free and total testosterone serum concentration levels, such as a testosterone inhibiting agent.

Also included in the methods, kits, combinations, and compositions of the present invention are pharmaceutical compositions comprising a hormone disorder-effective amount of a pharmaceutical agent for decreasing sex hormone-binding globulin serum concentration levels.

Also included in the methods, kits, combinations, and compositions of the present invention are pharmaceutical compositions comprising a hormone disorder-effective amount of a pharmaceutical agent for increasing serum insulin concentration levels.

Also included in the methods, kits, combinations, and compositions of the present invention are pharmaceutical compositions comprising a hormone disorder-effective amount of a pharmaceutical agent for increasing free and total estrogen serum concentration levels.

Also included in the methods, kits, combinations, and compositions of the present invention are pharmaceutical compositions comprising a hormone disorder-effective amount of a pharmaceutical agent for increasing free and total testosterone serum concentration levels.

Also included in the methods, kits, combinations, and compositions of the present invention are pharmaceutical compositions comprising a hormone disorder-effective amount of a pharmaceutical agent for increasing sex hormone-binding globulin serum concentration levels.

Additionally, the present invention optionally include the salts, esters, amides, enantiomers, isomers, tautomers, prodrugs, or derivatives of the therapeutic agents of the present invention, as well as emollients, stabilizers, antimicrobials, fragrances, and propellants.

The methods, kits, combinations, and compositions of the present invention provide enhanced treatment options for treating, preventing, or reducing the risk of developing a hormone disorder, and/or a disease or disorder associated with, or related to, a hormone disorder in a mammal, as compared to treatment options currently available.

The use of the term “about” in the present disclosure means “approximately.” The use of the term “about” also indicates that dosages outside the cited ranges may also be effective and safe, and such dosages are also encompassed by the scope of the present claims.

The phrase “pharmaceutically acceptable” is used adjectivally herein to mean that the modified noun is appropriate for use in a pharmaceutical product or therapy regime. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including, in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

The term “treat” or “treatment” as used herein refers to any treatment of a mammalian condition, disorder, or disease associated with, or related to, a hormone disorder, and includes, but is not limited to, preventing the condition, disorder, or disease from occurring in a mammal that may be predisposed to the condition, disorder, or disease, but has not yet been diagnosed as having the condition, disorder, or disease; inhibiting the condition, disorder, or disease, for example, arresting the development of the condition, disorder, or disease; relieving the condition, disorder, or disease, for example, causing regression of the condition, disorder, or disease; or relieving the condition caused by the disease or disorder, for example, stopping or relieving the symptoms of the disease or disorder. In one embodiment of the present invention, the condition, disorder, or disease associated with, or related to, a hormone disorder is cardiovascular disease.

The term “prevent” or “prevention,” in relation to a hormone disorder or disease, means no hormone disorder or disease development if none had occurred, or no further hormone disorder or disease development if there had already been development of a hormone disorder or disease.

The phrase “hormone disorder” refers to a condition, disorder, or disease that occurs in a mammal due to overproduction and/or underproduction of insulin, estrogen, testosterone, and/or sex hormone-binding globulin, and is associated with, or related to, serum concentrations of insulin, estrogen, testosterone, and/or sex hormone-binding globulin that are below or above that of a young healthy disease-free mammal.

As used herein, the phrase “hormone deficiency” refers to lower serum concentration levels of insulin, estrogen, testosterone, and/or sex hormone-binding globulin in a mammal as compared to the median serum levels for a young healthy disease-free mammal.

The phrase “estrogen deficient disorder” is used herein to describe a condition, disorder, or disease that occurs in a mammal due to lack of endogenous estrogen production. Such conditions, disorders, or diseases include, but are not limited to, hypogonadism, sexual dysfunction, decreased libido, hypercholesterolemia, abnormal electrocardiograms, vasomotor symptoms, diabetic retinopathy, hyperglycemia, hyperinsulinemia, hypoinsulinemia, increased percentage of body fat, hypertension, obesity, osteoporosis, osteopenia, vaginal dryness, thinning of the vaginal wall, menopausal symptoms and hot flashes, cognitive dysfunction, cardiovascular disease, Alzheimer's disease, dementia, cataracts, and cancer, including, but not limited to, cervical, uterine, or breast cancer.

The phrase “testosterone deficient disorder” is used herein to describe a condition, disorder, or disease that occurs in a mammal due to lack of endogenous testosterone production. Such conditions, disorders, or diseases include, but are not limited to, hypogonadism, sexual dysfunction, decreased libido, hypercholesterolemia, abnormal electrocardiograms, vasomotor symptoms, diabetic retinopathy, hyperglycemia, hyperinsulinemia, hypoinsulinemia, increased percentage of body fat, hypertension, obesity, osteoporosis, osteopenia, vaginal dryness, thinning of the vaginal wall, menopausal symptoms and hot flashes, cognitive dysfunction, cardiovascular disease, Alzheimer's disease, dementia, cataracts, and cancer, including, but not limited to, cervical, uterine, or breast cancer.

The phrase “sex hormone-binding globulin deficient disorder” is used herein to describe a condition, disorder, or disease that occurs in a mammal due to lack of endogenous sex hormone-binding globulin production.

A “hormone disorder-effective amount” is intended to qualify the amount of a therapeutic agent administered to a mammal that is required to treat or prevent a hormone disorder, or a disease or disorder associated with, or related to, a hormone disorder in the mammal; or relieve to some extent one or more of the symptoms associated with, or related to, a hormone disorder, or a disease or disorder associated with, or related to, a hormone disorder in the mammal. A hormone disorder-effective amount includes, but is not limited to, administering an amount of a therapeutic agent that achieves, or maintains, a therapeutic F factor in the serum of a mammal. In one embodiment of the present invention, the F factor is compared to a graph plotting the Free Insulin Testosterone Index in a male and/or female subject, for example see FIG. No. 10 to determine if the mammal has a hormone disorder or is at risk of developing a hormone disorder. From this graph a dose of a therapeutic agent is determined to achieve a therapeutic F factor in the mammal having been identified as having a hormone disorder, or at risk of developing a hormone disorder. The therapeutic agent is then administered at the determined dose, and the method is repeated until a therapeutic F factor is achieved in the mammal. Therapeutic agents useful in the present invention, include, but are not limited to: estrogen; testosterone; sex hormone-binding globulin; insulin; a pharmaceutical agent that increases the serum concentration of estrogen, testosterone, sex hormone-binding globulin and/or insulin in the mammal; and/or a pharmaceutical agent that decreases the serum concentration of estrogen, testosterone, sex hormone-disorder globulin, and/or insulin in the mammal; and/or all salts, esters, amides, enantiomers, isomers, tautomers, prodrugs and derivatives of these compounds or agents.

The therapeutic agents of the present invention are used in a “hormone disorder-effective amount.” This means that the amount of estrogen; testosterone; sex hormone-binding globulin; insulin; a pharmaceutical agent that increases the serum concentration of estrogen, testosterone, sex hormone-binding globulin and/or insulin in the mammal; and/or a pharmaceutical agent that decreases the serum concentration of estrogen, testosterone, sex hormone-disorder globulin, ink and/or insulin in the mammal; and/or all salts, esters, amides, enantiomers, isomers, tautomers, prodrugs and derivatives of these compounds or agents; administered to a mammal is such that a therapeutic dose is delivered over the term that the drug is to be used. A therapeutic dose achieves or maintains a therapeutic F factor in the mammal over the course of the treatment. Such delivery is dependent on a number of variables including the type of formulation used, the time period for which the individual dosage unit is to be used, the flux rate of the therapeutic agent from the formulation, surface area of application site, etc. The amount of therapeutic agent necessary can be experimentally determined by methods known in the art. It is understood, however, that specific dose levels of the therapeutic agents of the present invention for any particular patient depends upon a variety of factors including the activity of the specific compound employed, the age, body weight, general health, sex, and diet of the patient, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. Studies in animal models generally may be used for guidance regarding effective dosages for treatment of a hormone disorder in accordance with the present invention. In terms of treatment protocols, it should be appreciated that the dosage to be administered will depend on several factors, including the particular agent that is administered, the route administered the condition of the particular patient, etc. Generally speaking, one will desire to administer an amount of the compound that is effective to achieve a serum level commensurate with the concentrations found to be effective in vitro. Thus, where a compound is found to demonstrate in vitro activity at, for example, 10 ng/ml, one will desire to administer an amount of the drug that is effective to provide about a 10 ng/ml concentration in vivo. Determination of these parameters is well within the skill of the art. These considerations, as well as effective formulations and administration procedures are well known in the art and are described in standard textbooks.

Serum insulin concentration in a mammal can be measured by methods known in the art, for example, by double extraction technique.

Total serum estrogen in a mammal can be measured by assays known in the art, such as an ammonium sulfate precipitation assay (see, for example, Nankin et al., J. Clin. Endocrinol. Metab. 1975 Aug; 41(2):271-81), or a radioimmunoassay (see, for example, Furuyama et al., (1975)). Total serum estrogen refers to the sum of the free estrogen (that is, estrogen unattached to any protein), estrogen weakly bound to serum proteins, such as albumin-bound estrogen, and estrogen tightly bound to high affinity serum proteins, such as sex hormone-binding globulin-bound estrogen.

The serum levels of estrogen required for clinical efficacy are in the range of between 40-60 pg/ml. This range of values is the physiologic serum level of the pre-menopausal women during the early follicular phase.

Free serum testosterone levels in a mammal are measured by the recently validated and highly sensitive equilibrium dialysis method discussed in Sinha-Hikim et al., Clinical Endocrinology & Metabolism 1312-18. (1998). Total serum testosterone can be measured by assays such as a radioimmunoassay, see, for example, Furuyama et al., Steroids, 16:415-428 (1970).

For example, normal cycling women produce approximately 300 μg of testosterone per day. Their total serum testosterone levels generally range from about 20 ng/dL to about 80 ng/dL averaging about 40 ng/dL. In healthy young women, for example, mean free testosterone levels are generally about 3.6 pg/mL. However, several factors may influence both total and free testosterone serum levels. For example, in regularly ovulating women, there is a small but significant increase in plasma testosterone levels during the middle third of the menstrual cycle. However, mean testosterone levels (1.2 nmol/L or 33 ng/dL) and mean free testosterone levels (12.8 pmol/L or 3.6 pg/mL) during the luteal and follicular phases are not significantly different. Additionally, testosterone production declines continuously after age 30 so that serum testosterone levels in a 60-year-old woman are only 50% of the levels in a 30-year-old woman. Although the percentage of free testosterone generally does not vary with age, an absolute decline in free testosterone has been observed. This decline does not occur abruptly at menopause but instead occurs gradually and continuously as a result of the age-related decrease in both the adrenal and ovarian androgen production. Also, for example, after an ovariectomy, testosterone concentrations decrease by about 50%.

A variety of methods have been used to quantify the serum concentrations of sex hormone-binding globulin, including ammonium sulfate precipitation, gel filtration, equilibrium dialysis, dextran-coated charcoal, and radioimmunoassay (see, for example, Kahn et al., Radioimmunoassay for Human Testerone-Estradiol Binding Globulin, J Clinical Endocrinology and Metabolism, Vol. 54:705-710 (1982). The mean serum sex hormone-binding globulin level in healthy pre-menopausal women is about 84 nmol/liter and the normal range is about 36 nmol/liter to about 185 nmol/liter. Serum sex hormone-binding globulin levels are known to be elevated in women treated with oral estrogens, estrogen-containing oral contraceptives, clomiphene, tamoxifen, raloxifene, phenytoin, and sodium valproate, as well as in women who are pregnant, hyperthyroid, have chronic liver disease and HIV infection; see, for example, Bond et al., Sex Hormone-binding Globulin in Clinical Perspective, Acta. Obstet. Gynecol. Scand., Vol. 66:255-262 (1987). See also, for example, Miller et al. (1998). In healthy men, the mean sex hormone-binding globulin serum level is generally less than about 10 nmol/liter. Serum levels of sex hormone-binding globulin increase with age to about 50 nmol/liter to about 60 nmol/liter or greater.

Toxicity and therapeutic efficacy of the therapeutic agents of the present invention can be determined by standard pharmaceutical procedures,for example, for determining LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic induces are preferred. While compounds that exhibit toxic side effects may be used, care should be taken to design a delivery system that targets such compounds to the site of affected tissue in order to minimize potential damage to unaffected cells and, thereby, reduce side effects.

The therapeutic agents of the present invention may be administered, if desired, in the form of salts, esters, amides, enantiomers, isomers, tautomers, prodrugs, derivatives and the like, provided the salt, ester, amide, enantiomer, isomer, tautomer, prodrug, or derivative is suitable pharmacologically, that is, effective in the present methods, combinations and compositions. Salts, esters, amides, enantiomers, isomers, tautomers, prodrugs and other derivatives of the active agents may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992). For example, acid addition salts are prepared from the free base using conventional methodology, and involves reaction with a suitable acid. Generally, the base form of the drug is dissolved in a polar organic solvent such as methanol or ethanol and the acid is added thereto. The resulting salt either precipitates or may be brought out of solution by addition of a less polar solvent. Suitable acids for preparing acid addition salts include both organic acids, for example, acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, for example, hydrochloric acid, hydrobromic acid, sulfuric acid, nitric acid, phosphoric acid, and the like. An acid addition salt may be reconverted to the free base by treatment with a suitable base. Particularly preferred acid addition salts of the active agents herein are halide salts, such as may be prepared using hydrochloric or hydrobromic acids. Conversely, preparation of basic salts of acid moieties which may be present on a phosphodiesterase inhibitor molecule are prepared in a similar manner using a pharmaceutically acceptable base such as sodium hydroxide, potassium hydroxide, ammonium hydroxide, calcium hydroxide, trimethylamine, or the like. In one embodiment, basic salts are alkali metal salts, for example, the sodium salt, and copper salts. Preparation of esters involves functionalization of hydroxyl and/or carboxyl groups which may be present within the molecular structure of the drug. The esters are typically acyl-substituted derivatives of free alcohol groups, that is, moieties that are derived from carboxylic acids of the formula RCOOH where R is alkyl, and in one embodiment is lower alkyl. Esters can be reconverted to the free acids, if desired, by using conventional hydrogenolysis or hydrolysis procedures. Amides and prodrugs may also be prepared using techniques known to those skilled in the art or described in the pertinent literature. For example, amides may be prepared from esters using suitable amine reactants, or they may be prepared from an anhydride or an acid chloride by reaction with ammonia or a lower alkyl amine. Prodrugs are typically prepared by covalent attachment of a moiety, which results in a compound that is therapeutically inactive until modified by an individual's metabolic system.

The therapeutic agents of the present invention can be formulated as a single pharmaceutical composition or as independent multiple pharmaceutical compositions. Pharmaceutical compositions according to the present invention include those suitable for oral, percutaneous, transmucosal, implantation, inhalation spray, rectal, vaginal, topical, buccal (for example, sublingual), or parenteral (for example, subcutaneous, intramuscular, intravenous, intramedullary and intradermal injections, or infusion techniques) administration, although the most suitable route in any given case will depend on the nature and severity of the condition being treated and on the nature of the particular compound which is being used. Illustratively, methyltestosterone is administered orally, and testosterone and estradiol are administered percutaneously.

For oral administration, the pharmaceutical composition of the therapeutic agents may be in the form of, for example, a tablet, capsule, cachet, lozenge, dispensable powder, granule, solution, suspension, emulsion or liquid. Capsules, tablets, etc., can be prepared by conventional methods well known in the art. Oral formulations can contain excipients such as binders (for example, hydroxypropylmethylcellulose, polyvinyl pyrilodone, other cellulosic materials and starch), diluents (for example, lactose and other sugars, starch, dicalcium phosphate and cellulosic materials), disintegrating agents (for example, starch polymers and cellulosic materials) and lubricating agents (for example, stearates and talc). Solutions, suspensions and powders for reconstitutable delivery systems include vehicles such as suspending agents (for example, gums, zanthans, cellulosics and sugars), humectants (for example, sorbitol), solubilizers (for example, ethanol, water, PEG and propylene glycol), surfactants (for example, sodium lauryl sulfate, Spans, Tweens, and cetyl pyridine), preservatives and antioxidants (for example, parabens, vitamins E and C, and ascorbic acid), anti-caking agents, coating agents, and chelating agents (for example, EDTA).

Percutaneous administration includes transdermal delivery systems that include patches, gels, tapes and creams, and can contain excipients such as alcohols, penetration enhancers, and thickeners, as well as solubilizers (for example, propylene glycol, bile salts, and amino acids), hydrophilic polymers (for example, polycarbophil and polyvinylpyrolidone), and adhesives and tackifiers (for example, polyisobutylenes, silicone-based adhesives, acrylates and polybutene).

Transmucosal formulations or delivery systems include patches, tablets, suppositories, pessaries, gels and creams, and can contain excipients such as solubilizers and permeation enhancers (for example, propylene glycol, bile salts and amino acids), and other vehicles (for example, polyethylene glycol, fatty acid esters and derivatives, and hydrophilic polymers such as hydroxypropylmethylcellulose and hyaluronic acid).

Injectable drug formulations include solutions, suspensions, gels, microspheres and polymeric injectables, and can comprise excipients such as solubility-altering agents (for example, ethanol, propylene glycol and sucrose) and polymers (for example, polycaprylactones and PLGA's).

Implantable formulations or systems include rods and discs, and can contain excipients such as PLGA and polycaprylactone.

The therapeutic agents of the present invention can then be administered orally, percutaneously, transmucosally, by implantation, by inhalation spray, rectally, vaginally, topically, buccally or parenterally in dosage unit formulations containing conventional nontoxic pharmaceutically acceptable carriers, adjuvants, and vehicles as desired. The compounds of the present invention can be administered by any conventional means available for use in conjunction with pharmaceuticals, either as individual therapeutic compounds or as a combination of therapeutic compounds.

The compositions of the present invention can be administered for the prophylaxis or treatment of a hormone disorder by any means that produce contact of these compounds with their site of action in the body, for example in the ileum, the plasma, or the liver of a mammal.

Additionally, the methods, combinations and compositions of the present invention may optionally include salts, emollients, stabilizers, antimicrobials, fragrances, and propellants.

In another embodiment of the present invention, the therapeutic agents come in the form of kits or packages. The therapeutic agents of the present invention can be packaged in the form of kits or packages in which the daily (or other periodic) dosages are arranged for proper sequential or simultaneous administration. The present invention further provides a kit or package containing a plurality of dosage units, adapted for successive daily administration, each dosage unit comprising at least one of the therapeutic agents of the present invention. This drug delivery system can be used to facilitate administering any of the various embodiments of the therapeutic compositions. The kits or packages also contain a set of instructions for the patient.

The present methods, kits, combinations, and compositions can also be used in combination therapy with another pharmaceutical agent that treats a hormone disorder, or a disease or disorder associated with, or related to, a hormone disorder; or the symptoms related to, or associated with, a hormone disorder, or a disease or disorder associated with, or related to, a hormone disorder.

The phrase “combination therapy” embraces the administration of estrogen; testosterone; sex hormone-binding globulin; insulin; a pharmaceutical agent that increases the serum concentration of estrogen, testosterone, sex hormone-binding globulin and/or insulin in the mammal; and/or a pharmaceutical agent that decreases the serum concentration of estrogen, testosterone, sex hormone-disorder globulin, and/or insulin in the mammal; and/or all salts, esters, amides, enantiomers, isomers, tautomers, prodrugs and derivatives of these compounds or agents; as part of a specific treatment regimen intended to provide a beneficial effect from the co-action of these therapeutic agents for the treatment of a hormone disorder, or a disease or disorder associated with, or related to, a hormone disorder in a mammal, or the symptoms associated with, or related to, a hormone disorder, or a disease or disorder associated with, or related to, a hormone disorder. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days, weeks, months or years depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, where each therapeutic agent is administered at a different time, as well as administration of these therapeutic agents, or at least two of the therapeutic agents, in a substantially simultaneous manner. Substantially simultaneous administration can be accomplished, for example, by administering to the subject a single gel having a fixed ratio of each therapeutic agent or in multiple, single capsules, tablets, or gels for each of the therapeutic agents. Sequential or substantially simultaneous administration of each therapeutic agent can be effected by any appropriate route including, but not limited to, oral routes, percutaneous routes, intravenous routes, intramuscular routes, and direct absorption through mucous membrane tissues. The therapeutic agents can be administered by the same route or by different routes. For example, a first therapeutic agent of the combination selected can be administered orally, while the other therapeutic agents of the combination can be administered percutaneously. Alternatively, for example, all therapeutic agents may be administered percutaneously, or all therapeutic agents may be administered intravenously, or all therapeutic agents may be administered intramuscularly, or all therapeutic agents can be administered by direct absorption through mucous membrane tissues. The sequence in which the therapeutic agents are administered is not narrowly critical. “Combination therapy” also can embrace the administration of the therapeutic agents as described above in further combination with other biologically active ingredients, such as, but not limited to, agents for treating a hormone disorder, or a disease or disorder associated with, or related to, a hormone disorder, such as, but not limited to, cardiovascular disease, and non-drug therapies, such as, but not limited to, surgery.

The therapeutic compounds which make up the combination therapy may be a combined dosage form or in separate dosage forms intended for substantially simultaneous oral administration. The therapeutic compounds that make up the combination therapy may also be administered sequentially, with either therapeutic compound being administered by a regimen calling for two-step administration. Thus, a regimen may call for sequential administration of the therapeutic compounds with spaced-apart administration of the separate, active agents. The time period between the multiple administration steps may range from, for example, a few minutes to several hours to days, depending upon the properties of each therapeutic compound such as potency, solubility, bioavailability, plasma half-life and kinetic profile of the therapeutic compound, as well as depending upon the effect of food ingestion and the age and condition of the patient. Circadian variation of the target molecule concentration may also determine the optimal dose interval. The therapeutic compounds of the combined therapy whether administered simultaneously, substantially simultaneously, or sequentially, may involve a regimen calling for administration of one therapeutic compound by oral route and another therapeutic compound by percutaneous route. Whether the therapeutic compounds of the combined therapy are administered orally, by inhalation spray, rectally, topically, buccally (e.g., sublingual), or parenterally (e.g., subcutaneous, intramuscular, intravenous and intradermal injections, or infusion techniques), separately or together, each such therapeutic compound will be contained in a suitable pharmaceutical formulation of pharmaceutically-acceptable excipients, diluents or other formulations components. Examples of suitable pharmaceutically-acceptable formulations containing the therapeutic compounds are given above. Additionally, drug formulations are discussed in, for example, Hoover, John E., Remington's Pharmaceutical Sciences, Mack Publishing Co., Easton, Pa. 1975. Another discussion of drug formulations can be found in Liberman, H. A. and Lachman, L., Eds., Pharmaceutical Dosage Forms, Marcel Decker, New York, N.Y., 1980.

Additionally, testosterone has little or no interaction with sulfonylureas, biguanides and insulin, making it suitable for combination therapy without concern of interaction with these drugs. In clinical application, testosterone can be added and, in time, the other agents discontinued or reduced. Testosterone at the present time is used to treat hypogonadism, the absence or lack of testosterone.

The present invention is further illustrated by the following examples, which should not be construed as limiting in any way.

### EXAMPLES

### Example 1

C. W. was a sixty-five-year-old white male with a fifteen-year history of insulin dependent diabetes mellitus. He was hospitalized with gangrene of the middle finger of his right hand and was scheduled for amputation. He had already had bilateral femoral-popliteal bypass surgery for vascular insufficiency of the lower extremities. Due to anorexia, he was given an intramuscular injection of 200 mg of aqueous testosterone. His fasting glucose fell from 212 to 159. Repeated dosing every other day for one week resulted in a normalization of his serum glucose and a decrease in his insulin requirement from 44 units of Humulin (insulin, Lilly) daily to 24 units. His finger healed completely in two months making amputation unnecessary.

### Example 2

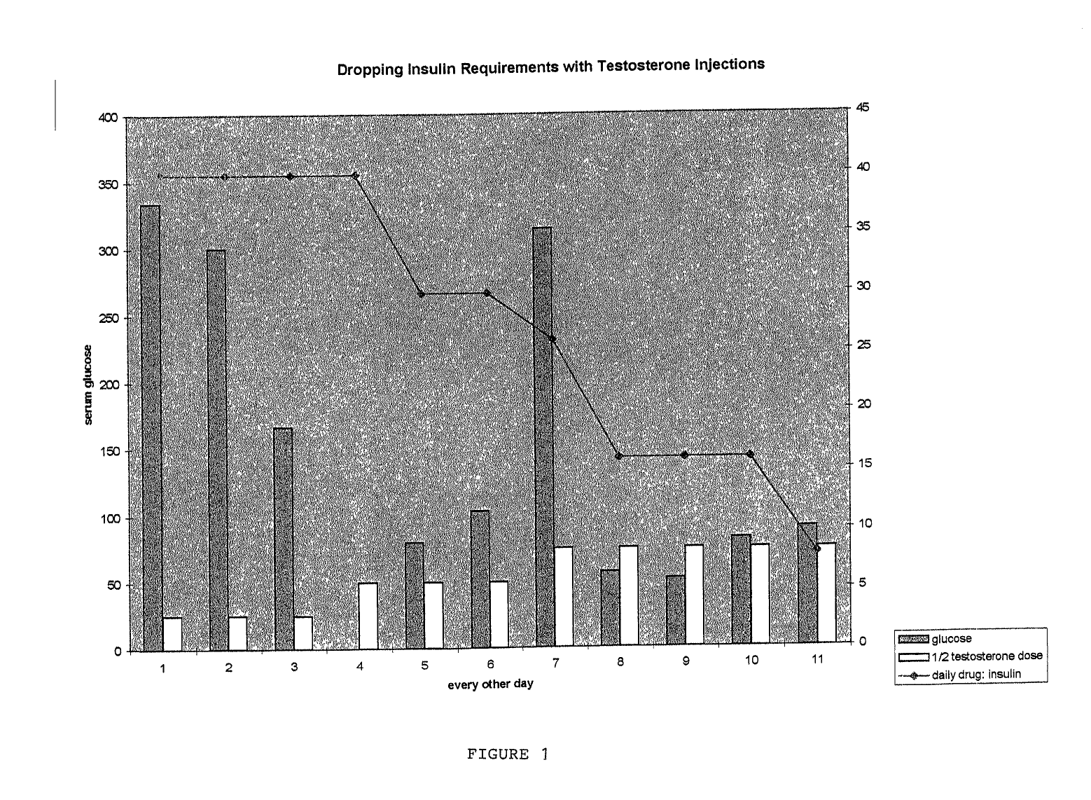

A. W. was a seventy-four-year-old black female who has been an insulin dependent diabetic for fifteen years. She had two toes removed previously for gangrene on the left foot; the remaining toes showed dry gangrene and a draining ulceration. She was scheduled for amputation. She used 24 units of Humulin insulin in the morning and 16 units at night. Her fasting blood glucose was 334. Treated with 150 mg of depo-testosterone enanthate intramuscularly, three times weekly, she became euglycemic on less than 12 units daily; see FIG. 1. However, due to the infected heel ulcer, amputation proceeded.

### Example 3

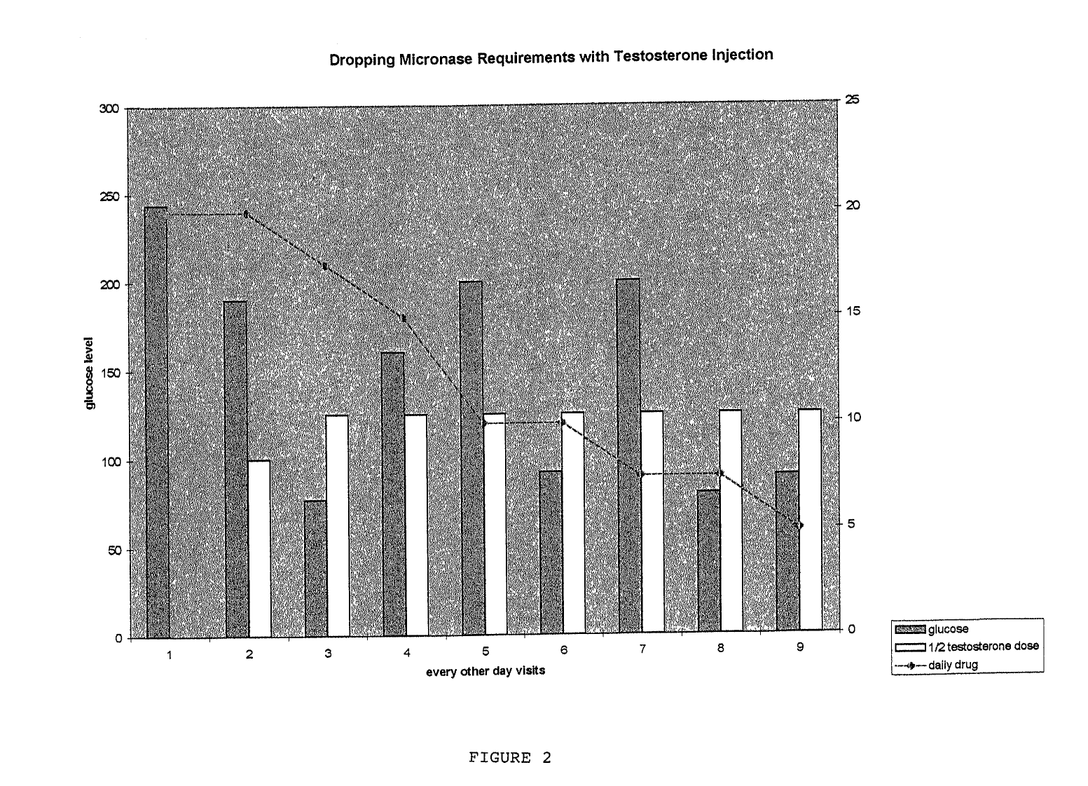

J. G. was an eighty-one-year-old white male on oral hypoglycemic agents for ten years. Present dosage was 20 mg/day, Micronase 7 (Upjohn). He presented with ulcerations in four areas of his left foot, no dorsal pedis pulsation, and was scheduled for amputation but refused. Fasting glucose was 244. Treatment with 250 mg depo-testosterone enthanale intramuscularly three times weekly for four weeks resulted in J. G. becoming euglycemic and discontinuing his oral agents, see FIG. 2.

### Example 4

G. S. was a forty-three-year-old white male with uncontrolled diabetes for ten years. Seen intermittently for ulceration of the feet, he presented with a serum glucose of 550 while on 100 units of Humulin. He refused admission to the hospital.

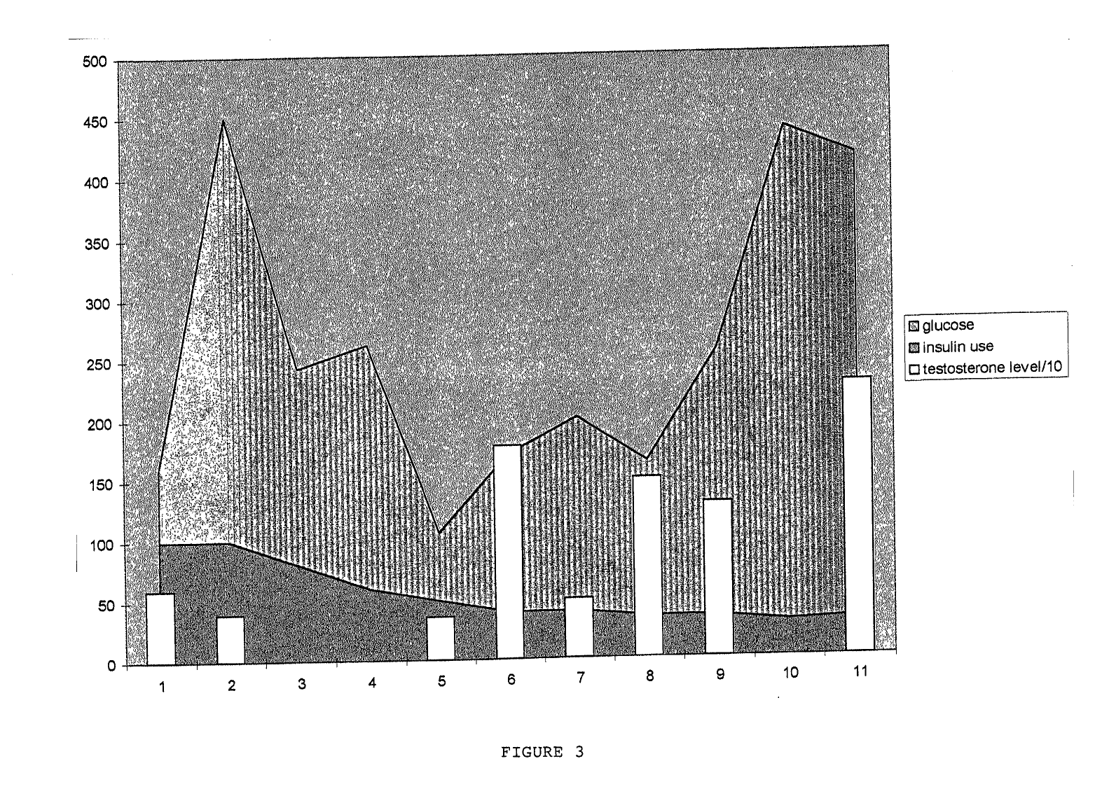

Over the course of six months, he received 250 to 900 mg of aqueous testosterone subcutaneous pellets every six (6) weeks. His insulin requirements were reduced 35 units while his serum glucose remained stable at 140 to 180 mg/dl; see FIG. 3.

### Example 5

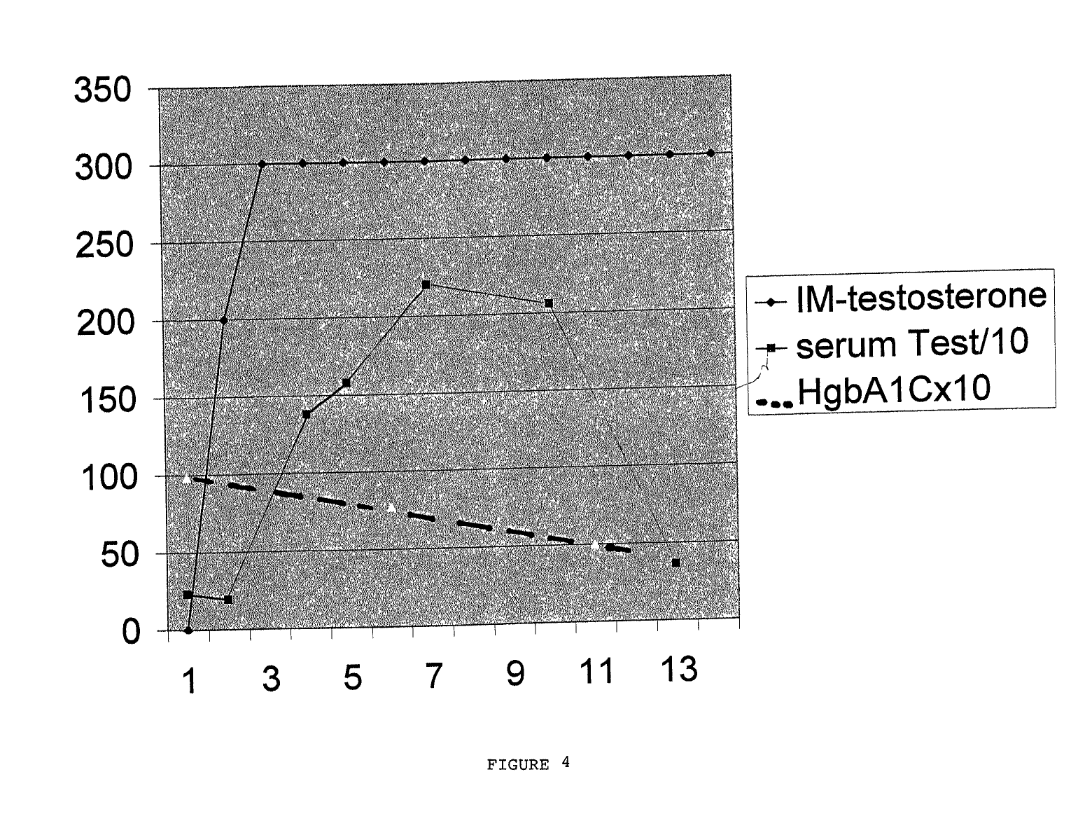

Testosterone enanthate 300 mg was given to a subject intramuscularly twice weekly for four weeks, then continued with therapy of 300 mg every two weeks. The subject's Hemoglobin A1C dropped from 9.9 in May, 1998 to 7.2 in August, 1998 to 5.5 in October, 1998 demonstrating a drop in tissue glucose stores as shown in FIG. 4.

### Example 6

A subset of 161 post-menopausal women were identified from the 461 women in the A.L.A.R.M. (Association for Lipids and Atherosclerosis Research in Michigan, 1995-1996) database. Both the 49 using estrogen supplements and the remaining 112 not using estrogen supplements had samples of their serum analyzed for estradiol, total testosterone and free testosterone, dehydro-epiandrosterone sulfate, androstenedione, cortisol, insulin and sex hormone-binding globulin.

One hundred and sixty-one post-menopausal women were identified from the A.L.A.R.M. database. Included for each was a comprehensive cardiovascular risk database and anthropologic measurements of blood pressure, body mass index and waist-to-hip ratios. Serum lipid measurements were processed, while maintaining frozen serum in storage at −80° Centigrade. Radio immunoassay assays (see Table No. 1) were performed for estradiol, total testosterone, free testosterone, dehydro-epiandrosterone sulfate, androstenedione, sex-hormone-binding globulin, cortisol and insulin.

TABLE No. 1
Radio Immunoassay

| LABORATORY ASSAYS | | | COEFFICIENTS | |
|---|---|---|---|---|
| LAB TEST | SOURCE | MDD | Intra-assay | Inter-assay |
| | Coated Tube | | | |
| | Immuno- | | | |
| | radiometric | | | |
| | Assay | | | |
| Testosterone | Diagnostic | 2.1 ug/dl | C.V = 6.4% | C.V. = 9.5% |
| Total | Product Corp | | | |
| Testosterone | Diagnostic | 0.18 pg/mlP | C.V. = 7.3% | Single assay |
| Free | Product Corp | | | |
| Dehydro-epian | Diagnostic | 2.1 ug/dl | C.V. = 6.4% | C.V. = 9.5% |
| drosterone | Product Corp | | | |
| Sulfate | | | | |
| Cortisol | Diagnostic | 0.2 ug/dl | C.V. = 6.4% | Single assay |
| | Product Corp | | | |
| | Double | | | |
| | Antibody | | | |
| | Technique | | | |
| Insulin | Diagnostic | 1.3 mIU/ml | C.V. = 4.6% | Single assay |
| | System Labs | | | |
| Estradiol | Diagnostic | 1.4 pg/ml | C.V. = 7.3% | C.V. = 1.3% |
| | System Labs | | | |
| Androstere- | Diagnostic | 0.02 ng/ml | C.V. = 7.5% | Single assay |
| dione | System Labs | | | |
| | Coated Tube | | | |
| | Immuno- | | | |
| | radiometric | | | |
| | Assay | | | |
| Sex Hormone- | Diagnostic | 3 nmol/L | C.V. = 7/8% | Single assay |
| Binding | System Labs | | | |
| Globulin | | | | |

Anthropologic measurements were performed in triplicate for future determination of body mass index and waist-to-hip ratios. Blood pressure readings were taken in triplicate and averaged using a sphygmomanometer to the nearest digit on the right arm of the seated participant after at least a 5-minute rest period. Diabetes was defined as having a previous history of being treated with insulin or a hypoglycemic medication. Heart disease was based on the physician's record of angina associated with changes in EKG or hospitalization/heart catheterization studies. Smoking was also considered as a co-variable in the statistical analysis.

The method for measuring estradiol has been described previously. Serum cholesterol and HDL cholesterol were determined by enzymatic procedure. Insulin was measured by double extraction technique. However, although the patient's last meal was reported as the day before the evaluation, the time of day at which the blood was drawn varied from 8:00 AM to 4:30 PM. It is unlikely that the time of assay led to any systematic bias in the association between sex hormones and cardiovascular risk factors.

All statistical analyses were performed with SPSS version 6.1. In all analyses, a two-tailed value of P less than or equal to 0.05 was considered significant. In the multiple-regression model used to determine the relationship of sex hormones and risk factors for cardiovascular disease, the risk factors for cardiovascular disease were the dependent variables and the hormonal parameters the independent variables.

Even though essentially all of the estrogen and much of the testosterone in women are derived from androstenedione, all non-testosterone hormonal parameters (estradiol, dehydro-epiandrosterone sulfate, androstenedione, and cortisol) were considered unbound for analysis. Their ratios to sex-hormone-binding globulin were also included in the analysis. To determine whether significant correlation existed between any two independent variables in the study, partial correlation coefficients were calculated after controlling for age and body mass index.

The mean for the variables measure in the total group of 161 women and the subgroup with estrogen (49) and without estrogen supplementation (112) are shown in Table No. 2.

The correlation coefficients for the most significant factors, body mass index, waist-to-hip ratio and Testosterone Ratio Test (total testosterone/sex hormone-binding globulin) appear in Table No. 3.

The correlation coefficients for the Testosterone Ratio Test (TRT) for: (1) the total group, (2) the subset with estrogen, and (3) the subset not taking estrogen appear in Table No. 4.

Independent hormonal correlation coefficients were as follows:

Testosterone was significantly positively correlated with dehydro-epiandrosterone sulfate (r=0.516, p<0.000) and free testosterone (r=0.945, p<0.000). The assay of free testosterone was significantly and positively correlated with androstenedione (r=.416, p<006).

Estradiol was significantly and inversely correlated with body mass index (r=-0.324, p<034) and waist-to-hip ratio (r=-0.313, p<0.038). Estradiol divided by testosterone ratio was significantly and inversely correlated with androstenedione (r=-0.429, p<0.014), body mass index (r=-0.387, p<0.028), dehydro-epiandrosterone sulfate (r=-0.367, p<0.035). Cortisol correlated significantly and directly with Androstenedione (r=0.407, p<000), and HDL (r=0.199, p<018) and inversely with body mass index (r=-0.265, p<0.001). Androstenedione correlated significantly and directly with dehydro-epiandrosterone sulfate, testosterone, and free testosterone.

Note that apoprotein-B did not correlate with any risk factors except the lipid profiles:

HDL/Cholesterol ratio (r=0.702, p<000), HDL (r=- 0.380, p<0.011), LDL (r=0.831, p<0.000), total cholesterol (0.805, p<0.000), and triglycerides (r=0.663, p<0.000). The HDL Cholesterol ratio correlated inversely with body mass index (r=-0.252, p<0.002), waist-to-hip ratio (r=-0.299, p<0.000), all lipid parameters (p<0.000) and dehydro-epiandrosterone sulfate (r=-0.282, p<0.022). The only direct correlation was to HDL correlation was positive (r=0.747, p<0.000).

Table No. 2 shows the mean and standard deviations for the parameter considered in the statistical analysis. There was no significant difference between estrogen and non-estrogen users with respect to years since menopause, dehydro-epiandrosterone sulfate, estradiol, total or free testosterone, total cholesterol, HDL, LDL, HDL/total cholesterol ratio, systolic and diastolic blood pressure, smoking and anthropologic measurements.

TABLE No. 2
Post-menopausal women: Mean and Standard Deviations

| Variable | Mean | Cases |
|---|---|---|
| Age | 60.6455 | 95 |
| body mass index | 31.1650 | 95 |
| DM | 1.8421 | 95 |
| Testosterone | 0.2422 | 95 |
| TC | 223.4947 | 95 |
| HDL | 0.2513 | 95 |

Table No. 3 shows the Pearson correlation coefficients between body mass index, waist-to-hip ratio, and Testosterone Ratio Test. The correlation between body mass index and waist-to-hip ratio showed strong positive correlation with multiple cardiovascular risk factors. The Testosterone Ratio Test showed as strong a positive correlation with these same factors. None of the independent hormonal parameters, alone, showed a significant correlation.

TABLE No. 3
Summary of the Principle Cardiovascular Risk Factors Pearson Correlation
Coefficients (‘r’ and ‘p’ values) for entire population

| | Body mass | | Waist-to- | | HDL/ | | Testosterone | |
|---|---|---|---|---|---|---|---|---|
| | index | | hip ratio | | Cholesterol | | Ratio Test | |
| Factor | r | p | r | p | r | p | r | p |
| Age | −.206 | .009 | .065 | NS | .032 | NS | −.107 | NS |
| Cortisol | −.265 | .001 | −.020 | NS | .064 | NS | −.103 | NS |
| Androstene- | .030 | N/A | .0176 | .026 | .001 | NS | .401 | .000 |
| dione | | | | | | | | |
| body mass | | | .366 | .000 | −.252 | .002 | .278 | .000 |
| index | | | | | | | | |
| BP diastolic | .240 | .005 | .207 | .012 | −.118 | NS | .128 | .128 |
| dehydro- | .135 | .094 | .014 | NS | −.182 | .002 | .278 | .000 |
| epiandro- | | | | | | | | |
| sterone | | | | | | | | |
| sulfate | | | | | | | | |
| Insulin | .359 | .000 | .162 | .040 | .038 | NS | .087 | .271 |
| HDL/CHOL | −.252 | .002 | −.299 | .000 | | | −.202 | .011 |
| HDL | −.353 | .000 | −.372 | .000 | .757 | .000 | −.196 | .014 |
| Triglycerides | .194 | .016 | .149 | .057N | −.431 | .000 | .185 | .020 |
| TRT | .401 | .000 | .211 | .007 | −.202 | 0.11 | | |
| Testosterone | .101 | .213 | .076 | .335N | −.107 | NS | .823 | .000 |
| ApoB | .159 | .308 | .239 | .123N | −.702 | .000 | .128 | .421 |
| sex | −.564 | .000 | — | .025 | −.291 | NS | −.377 | .025 |
| hormone- | | | | | | | | |
| binding | | | | | | | | |
| globulin | | | | | | | | |
| waist-to-hip | .366 | .000 | | | −.299 | NS | .211 | .007 |
| ratio | | | | | | | | |

Table No. 4 showed the Pearson correlation coefficients for the subset using and not using estrogen supplementation.

TABLE No. 4
Testosterone Ratio Test Risk Factors Pearson Correlation Coefficients
(‘r’ and ‘p’ values) Total group, Estrogen Users, Non-Estrogen Users

| | Testosterone | | Testosterone | | Testosterone | |
|---|---|---|---|---|---|---|
| | Ratio Test | | Ratio Test | | Ratio Test | |
| | (total = 161) | | (estrogen = 49) | | (no = 12) | |
| Factor | r | p | r | p | r | p |
| Age | −.107 | NS | −.285 | NS | −.059 | NS |
| Cortisol | −.103 | NS | −.257 | NS | .060 | NS |
| Androsteiedione | .401 | .000 | .297 | NS | .485 | .000 |
| body mass index | .278 | .000 | .164 | NS | .360 | .000 |
| BP diastolic | .128 | .128NS | .081 | NS | .171 | NS |
| dehydro-epiandro- | .406 | .000 | .500 | .001 | .386 | .000 |
| sterone sulfate | | | | | | |
| Insulin | .087 | .271NS | −.002 | NS | .096 | .271NS |
| HDL/CHOL | −.202 | .011 | −.291 | NS | −.173 | NS |
| HDL | −.196 | .014 | −.281 | NS | −.133 | NS |
| Triglycerides | .185 | .020 | .023 | NS | .360 | .000 |
| Testosterone | .823 | .000 | .937 | .000 | .681 | .000 |
| ApoB | .128 | .421NS | .128 | NS | .182 | NS |
| waist-to-hip ratio | .211 | .007 | .294 | .053NS | .184 | .053NS |
| sex hormone- | −.337 | .025 | −.337 | .025 | −.472 | .000 |
| binding globulin | | | | | | |

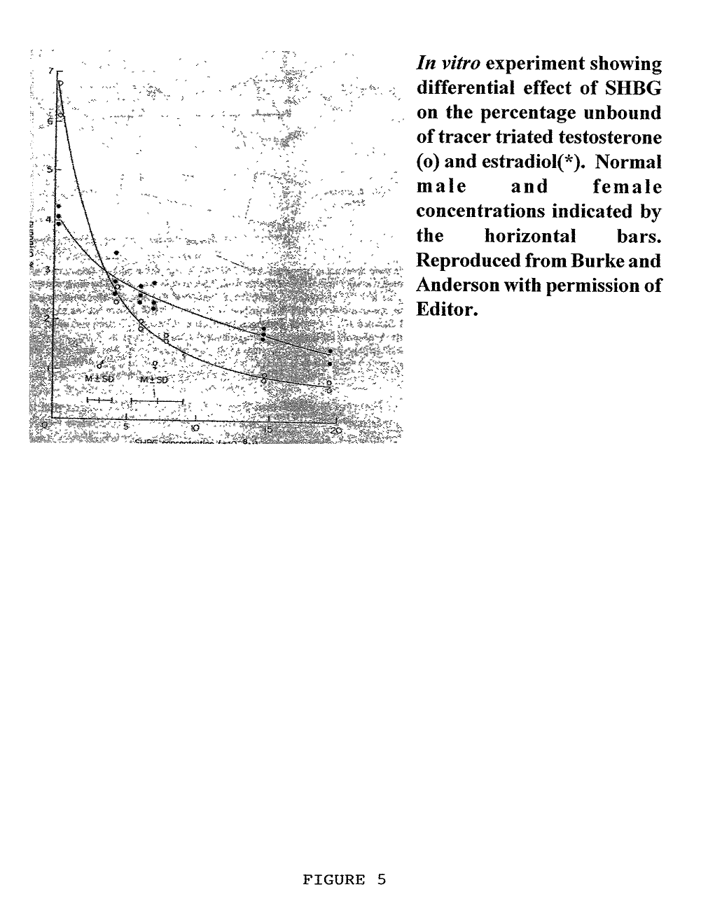

Table No. 5 outlines the correlation coefficients in those publications limited to post-menopausal women with covariant analysis of hormonal and cardiovascular risk factors. They reported striking but dissimilar results. This inconsistency may be explained by their omission of the calculated value of Testosterone Ratio Test. Based on Anderson's (1974) in vitro experiments, the percentage of unbound testosterone increases significantly with dropping sex hormone-binding globulin levels. See FIG. 5. But, in his experiment he kept the testosterone levels stable. In an in vivo study, the amount of unbound or free testosterone is influenced also by the amount of testosterone produced by the individual. Therefore, it is contemplated that the expected correlation of the Testosterone Ratio Test to cardiovascular risk factors is greater than either that of sex hormone-binding globulin or free testosterone levels.

TABLE No. 5
Correlation Coefficients

| | | Correlation- | Author/ |
|---|---|---|---|
| Hormone | Risk Factor | Coefficient | year |
| Free testosterone | Degree of CAD | p < .008 | Phillips, |
| | | | 1997 |
| sex hormone-binding | −body mass index | p < .001 | Haffner, |
| globulin | | | 1995 |
| sex hormone-binding | −glycosylated HgB | p < .001 | |
| globulin | | | |
| sex hormone-binding | −diastolic BP | p < .01 | |
| globulin | | | |
| sex hormone-binding | +HDL & HDL/TC | p < .001 | |
| globulin | | | |
| Free Testosterone | +HDL/TC | p < .05 | |
| Total Testosterone | +HDL/TC | p < .01 | |
| Total Testosterone | +body mass index | p < .01 | |
| Total Testosterone | +Systolic BP | p < .01 | |
| Total Testosterone | +Diastolic BP | p < .01 | |
| Estrone | −HDL/TC | p < .05 | |
| sex hormone-binding | +waist-to-hip ratio | p < .001 | Hauner, |
| globulin | | | 1995 |
| Testosterone | +serum insulinp | <.01 | |
| sex hormone-binding | −Triglycerides | p < .01 | Svendsen, |
| globulin | | | 1993 |
| sex hormone-binding | +waist-to-hip ratio | p < .001 | |
| globulin | | | |
| sex hormone-binding | +HDL | p < .05 | |
| globulin | | | |
| Androstenedione | −HDL | p < .05 | |
| Estradiol | −HDL | p < .05 | |
| Estradiol | −TC | p < .01 | |
| sex hormone-binding | +HDL | p < .001 | Haffner, |
| globulin | | | 1992 |
| sex hormone-binding | −Triglycerides | p < .05 | |
| globulin | | | |
| sex hormone-binding | −serum insulin | p < .001 | |
| globulin | | | |
| sex hormone-binding | +HDL | p < .07 | Soler, |
| globulin | −triglycerides | p < .002 | 1989 |
| Estrone | +triglycerides | p < .003 | |

Testosterone binding is of high affinity (K approximately 10⁹ M⁻¹), readily reversible at 37° C. and, in men, is nearly saturated since the molar concentration of sex hormone-binding globulin binding sites in adult male plasma is only marginally greater than the molar concentration of testosterone. In female plasma the sex hormone-binding globulin concentration is twofold higher and the testosterone concentration tenfold lower than in men and therefore most of the binding sites are unoccupied. Estradiol binds less well than testosterone to sex hormone-binding globulin, better than testosterone to albumin, and does not bind significantly to CBG.

Despite the small size of the unbound fraction of steroid hormones, it is contemplated that the unbound fraction and not the bound fraction, which is biologically active.

Mathematical Basis for Testosterone Ratio Test

*Unbound {free} testosterone is proportional
to total amount of testosterone.
% U ˜ concentration of [total testosterone]
*Unbound {free} testosterone is inversely
proportional to sex hormone-binding globulin.
% U ˜ 1/[sex hormone-binding globulin]
Therefore, Unbound Free testosterone.
% U ˜ [total testosterone]/sex hormone-binding globulin
% U ˜ Testosterone Ratio Test [Testosterone Ratio Test]

In Table No. 5, free estradiol against total estradiol, sex hormone-binding globulin and the Testosterone Ratio Test are provided.

As noted in Table No. 4, estrogen users have lost most of the established risk factors of cardiovascular heart disease. This may imply that: 1) these individuals have a decreased risk of cardiovascular risk factors, and/or 2) estrogen has cardioprotective effects, and/or 3) estrogen raises sex hormone-binding globulin lowering the Testosterone Ratio Test.

Although the original premise of androgenicity as a predictor of cardiovascular risk is confirmed by the representation of total testosterone in the calculation of the Testosterone Ratio Test, there may be other factors than testosterone and estrogen that independently relate to sex hormone-binding globulin. It is interesting to note that the Testosterone Ratio test shows an extremely high correlation (p<0.000) to all the androgens mentioned: dehydro-epiandrosterone sulfate, androstenedione, and free and total testosterone.

There is a positive association between the ratio of testosterone and sex hormone-binding globulin, the Testosterone Ratio Test, and recognized cardiovascular risk factors. This association was independent of age and remained significant even after adjustment for the influence of body mass index. It was of greater statistical significance than free testosterone, total testosterone or sex hormone-binding globulin taken individually. The mathematical calculation of Testosterone Ratio Test takes into account the total and free testosterone, overall androgenicity, and sex hormone-binding globulin. Of particular interest was the observation that in multiple linear regression analysis, Testosterone Ratio Test was more strongly related to cardiovascular risk factors than apoprotein B, lipids, blood pressure and insulin levels. In post-menopausal women, increased androgens remain associated with cardiovascular risk factors and this may be best considered by measurement of the Testosterone Ratio Test.

As a result of the study set forth above in Example 6, Applicants determined first by review of significance of various combinations of all hormonal parameters tested that the percentage of unbound testosterone was predictive of both cardiac and diabetic disease states in both Caucasian and African-American women. The Testosterone Ratio Test showed as great a predictive value as the Total Cholesterol/HDL-Cholesterol ratio as a risk factor for heart disease, yet the Testosterone Ratio Test is independent of age, which the TC/HCL is not.

Applicant then sought to apply the Testosterone Ratio Test to adult onset diabetics. The Testosterone Ratio Test appears to offer benefits over other common testing parameters for diabetes as it is stable throughout the day and need not be performed with individual fasting. It should be noted that, in comparison, the standard glucose tolerance test misses more than fifty percent of individuals who have impaired glucose tolerance.

With the significance of the Testosterone Ratio Test determined in men and women with diabetes, and women with heart disease, an extensive literature search was performed to determine if this same ratio was applicable to other medical conditions. Haffner, Lindstedt, and Hauner confirmed our observation without making reference to the underlying physiology. That is, the Testosterone Ratio Test correlates directly with insulin levels and insulin resistance.

Normal levels of testosterone for both sexes appear in Table No. 6. In the United States, total testosterone is measured in ng/dl and sex hormone-binding globulin in pmol/L. The conversion from ng/dl to pmol/L is 0.0347.

Therefore, the Testosterone Ratio Test is calculated thusly: TT×0.0347/sex hormone-binding globulin.

TABLE No. 6
Testosterone Levels

| | | Sex Hormone-binding | Testosterone |
|---|---|---|---|
| | Total | Globulin (sex hormone- | Ratio Test |
| Sex | Testosterone | binding globulin) | (TRT) |
| Male | >400 ng/dl | <20 pmo1/L | 0.70 to 1.2 |
| Female | <40 ng/dl | >40 pmo1/L | 0.01 to .035 |

In a study of twenty adult onset diabetic men on dialysis, the Testosterone Ratio Test was found to be 0.2 in eight of the ten men. The other two men were newly diagnosed and had levels of0.5.

In a study of thirty-five men and women with confirmed myocardial disease, all the men had Testosterone Ratio Test levels below 0.4.

The Testosterone Ratio Test had its components provide a screening tool for the recognition and treatment of insulin resistance, Syndrome X and for the application of testosterone replacement.

In two men with cluster headaches and a low Testosterone Ratio Test, testosterone supplementation to therapeutic levels at the normal male maximum resulted in a complete absence of cluster headaches/migraines for two years.

In normal men, a total testosterone concentration is greater than 400 mg/dl. Sex hormone-binding globulin less than 20, thus presenting a testosterone ratio normally between approximately 0.7 to 1.2.

For insulin-resistant diabetic men, the testosterone ratio is generally 0.05 and lower.

For normal women, the concentration total testosterone is generally less than 30 mg/dl. The concentration of sex hormone-binding globulin is generally greater than 40, and the ratio of two ranges is normally between 0.01 to 0.04.

For insulin-resistant diabetic women, the ratio is generally 0.06 and greater.

### Example 7

One hundred sixty-three post-menopausal women were identified from a multicenter cardiovascular disease database. Included for each was a comprehensive cardiovascular risk database and anthropologic measurements of blood pressure, body mass index and waist-to-hip ratios. Serum lipid measurements were processed under the direction of the director at a C.D.C.-approved laboratory, while maintaining frozen serum in storage at -80° Centigrade. A 2cc aliquot was used for duplicate batch hormone analysis. RAI assays were performed for estradiol, total testosterone, free testosterone, dehydro-epiandrosterone sulfate, androstenedione, sex hormone-binding globulin (sex hormone-binding globulin), cortisol and insulin. Estradiol, the most potent estrogen, was measured because it both correlates with estrone in post-menopausal women and because it has relatively stronger affinity for sex hormone-binding globulin. The method for measuring estradiol has been described previously. A subset of 25 patients had free estradiol, total estrogens and estradiol levels measured at Quest Laboratories in California. The laboratory assay, technique, supplier and coefficients of variances appear as in Example 6, above in Table No. 1.

Informed consent was obtained at the beginning of the examination, which included measurements of height and weight. Anthropologic measurements were performed in triplicate for calculations of body mass index and waist-to-hip ratios. Blood pressure readings were taken in triplicate and averaged using a sphygmomanometer to the nearest digit on the right arm of the seated participant after at least a 5-minute rest period. Diabetes was defined as having a previous history of being treated with insulin or a hypoglycemic medication or having fasting serum glucose above 126 mg/ml. Heart disease was based on the physician's record of angina or myocardial infarction associated with changes in EKG or hospitalization/heart catherization studies. Hypertension was based on the physician's record and the continued use of hypertensive medications. Smokers were removed from the co-variant analyses.

Serum cholesterol and HDL cholesterol were determined by enzymatic procedure.

Insulin was measured by double extraction technique. Although the patient's last meal was reported as the day before the evaluation, the time of day at which the blood was drawn varied from 8:00 AM to 4:30 PM. Thus, it is unlikely that the time of assay led to any systematic bias in the association between sex hormones and cardiovascular risk factors.

All statistical analyses were performed with SPSS version 6.1. In all analyzes, a two-tailed value of P<=.05 was considered significant. In the multiple regression model, the listed variables were analyzed comparing the groups of post-menopausal women with and without estrogen replacement. In the logarithmic regression used to determine the relationship of listed variables and risk factors for cardiovascular disease, all cardiovascular risk factors were grouped together and estradiol, sex hormone-binding globulin, insulin and HDL cholesterol were considered independent variables.

Although 90% of the estrogen and 98% of the testosterone in women are bound by sex hormone-binding globulin, they were considered 100% bound for statistical analysis. Various ratios to sex hormone-binding globulin were also included in the analysis. To determine whether significant correlation existed between any two independent variables in the study, partial correlation coefficients were calculated by linear regression analysis after controlling for age and body mass index.

There is no significant difference between the groups except in reference to the estradiol levels resulting from the supplementary estrogen replacement. The age, body mass index, waist-to-hip ratio, systolic and diastolic blood pressure, total testosterone, free testosterone, cortisol, dehydro-epiandrosterone sulfate, androstenedione, and various cholesterol measurements are comparable.

Means and standard deviations for the variables in the study appear in Table No. 7.

TABLE No. 7
Post-menopausal Women: Mean and Standard Deviations

| | ALL WOMEN | | | NON-ESTROGEN USERS | | |
|---|---|---|---|---|---|---|
| | | Standard | | | Standard | |
| Variable | Mean | Dev. | Cases | Mean | Dev. | Cases |
| Insulin | 11.2334 | 7.7102 | 160 | 12.411 | 8.0348 | 116 |
| sex hormone- | 143.0776 | 87.3985 | 161 | 115.0256 | 60.3077 | 117 |
| binding globulin | | | | | | |
| Estradiol | 43.8444 | 64.6287 | 162 | 19.5154 | 41.2115 | 117 |
| Estradiol x | 8537.33 | 15723.9 | 161 | 2390.171 | 6193.02 | 117 |
| sex hormone- | | | | | | |
| binding globulin | | | | | | |
| Testosterone | 0.2831 | 0.3604 | 162 | 0.2625 | 0.2103 | 117 |
| Testosterone/sex | 0.0027 | 0.0032 | 161 | 0.0030 | 0.0028 | 117 |
| hormone-binding | | | | | | |
| globulin | | | | | | |
| Testosterone-free | 0.9183 | 0.8377 | 161 | 0.8991 | 0.6139 | 117 |
| Cortisol | 13.5725 | 5.8470 | 160 | 12.8276 | 5.0179 | 116 |
| dehydro-epiandro- | 101.2190 | 59.8350 | 162 | 101.862 | 58.369 | 117 |
| sterone sulfate | | | | | | |
| Androstenedione | 0.8275 | 0.4208 | 160 | 0.8492 | 0.4203 | 117 |
| Triglycerides | 127.4724 | 70.5147 | 163 | 120.0924 | 67.6587 | 119 |
| Total Cholesterol | 229.3496 | 39.8326 | 163 | 226.9747 | 42.8289 | 119 |
| HDL Cholesterol | 55.3312 | 14.9166 | 163 | 53.7563 | 14.1613 | 119 |
| LDL/Cholesterol | 153.9448 | 37.1552 | 163 | 154.4622 | 40.4271 | 119 |
| HDL-C/ Chol | 0.2480 | 0.0076 | 163 | 0.2449 | 0.0078 | 119 |
| Apoprotein(B) | 115.4662 | 29.1764 | 163 | 115.0420 | 30.7341 | 119 |
| Systolic Blood | 132.5195 | 18.6205 | 145 | 133.0777 | 18.4576 | 103 |
| Pressure | | | | | | |
| Diastolic Blood | 80.8069 | 9.7616 | 145 | 81.4401 | 10.1131 | 103 |
| Pressure | | | | | | |
| Age | 59.96 | 10.96 | 163 | 60.38 | 11.36 | 125 |
| body mass index | 30.3031 | 6.8116 | 160 | 31.1058 | 11.36 | 115 |
| Waist/Hip Ratio | 0.8220 | 0.0735 | 161 | 0.8310 | 0.0074 | 125 |

The frequency table (Table No. 7) compares the demographics of the non-estrogen and estrogen users based on race. There is a predominance of estrogen users within the Caucasian population, with 40 women reporting estrogen use versus only 7 African-American women using estrogen (p<0.001). The non-estrogen replacement therapy user population is relatively equal at 63 Caucasian versus 58 African-American women. There is a predominance of diabetes, hypertension and the all-disease group in the non-estrogen replacement therapy group as compared to the estrogen replacement therapy group (p<0.005).

Frequency of race and disease states appears in Table No. 8.

TABLE No. 8
Frequency Table

| | estrogen | non-estrogen | |
|---|---|---|---|
| | replacement | replacement | |
| Population | therapy | therapy | p value |
| Women (172) | 49 | 117 | ** |
| Race | | | |
| ˜White | 40 (38.8%) | 63 (61.2%) | 0.001** |
| ˜Black | 7 (10.8%) | 58 (89.2%) | |
| Diabetes (172) | 1 (2.1%) | 19 (15.6%) | 0.005** |
| Smoker (171) | 7 (15.0%) | 13 (10.5%) | n.s. |
| Angina (169) | 3 (6.4%) | 17 (13.9%) | n.s. |
| Hypertension (169) | 15 (31.9%) | 61 (50.0%) | 0.025* |
| MI (169) | 1 (2.1%) | 4 (3.3%) | n.s. |
| All diseases (169) | 16 (34.0%) | 72 (59.0%) | 0.003** |

**Power .80 @ .05 Alpha

Table Nos. 9a and 9b list the Pearson correlation coefficients of the 21 variables for the estrogen users (Table No. 9a) and the non-estrogen users (Table No. 9b) in respect to estradiol, insulin and sex hormone-binding globulin. For both groups there is a significant inverse correlation with sex hormone-binding globulin for insulin, body mass index and waist-hip ratio P<0.02). There is a significant direct correlation with insulin for body mass index and waist-hip ratio (p<0.004). HDL-cholesterol is significantly and directly correlated with sex hormone-binding globulin (p<0.001) and inversely correlated with insulin (p<0.034).

In non-estrogen users there exists strong direct correlation with sex hormone-binding globulin for free-testosterone, triglycerides and systolic blood pressure. Triglycerides show a direct correlation and age an inverse correlation only for non-estrogen users. No such correlation is found in the estrogen users.

TABLE No. 9a
Pearson Correlation Coefficients 2-Tailed Significance; Estrogen Users N = 49

| | | | | | Sex Hormone- | |
|---|---|---|---|---|---|---|
| | | | | | Binding | |
| | Estradiol | | Insulin | | Globulin | |
| | Pearson | Sig. 2- | Pearson | Sig. 2- | Pearson | Sig. 2- |
| Factor | correl. | tailed | correl. | tailed | correl. | tailed |
| Insulin | −.291 | .055 | | | −.353* | .019 |
| Sex hormone-binding globulin | .172 | .265 | −.353* | .019 | | |
| Estradiol | | | −.291 | .265 | .172 | .265 |
| Estradiol x Sex hormone-binding | .774** | .000 | −.319* | .035 | .662** | .000 |
| globulin | | | | | | |
| Testosterone-total | −.177 | .245 | −.047 | .763 | −.197 | .201 |
| Testosterone/Sex hormone- | −.201 | .191 | −.002 | .662 | −.337* | .025 |
| binding globulin | | | | | | |
| Testosterone-Free | −.177 | .249 | −.001 | .993 | −.297 | .050 |
| Cortisol | .157 | .314 | −.049 | .752 | .255 | .095 |
| AndrosteneD | .177 | .262 | .037 | .811 | −.298 | .053 |
| DHEAS | .034 | .825 | −.035 | .821 | −.321* | .034 |
| Triglycerides | .088 | .571 | .051 | .743 | −.094 | .548 |
| Cholesterol | −.010 | .950 | −.089 | .570 | .070 | .657 |
| HDL- | .184 | .232 | −.323* | .034 | .532** | .000 |
| HDL/C Ratio | .196 | .202 | −.284 | .065 | .451** | .002 |
| LDL-Cholesterol | −.162 | .294 | .071 | .649 | −.203 | .191 |
| Apo-B | −.209 | .174 | .133 | .394 | −.132 | .400 |
| BP Systolic | .060 | .708 | .093 | .563 | .042 | .794 |
| BP Diastolic | −.164 | .298 | .148 | .355 | −.078 | .626 |
| Age | .056 | .715 | −.014 | .930 | .358* | .017 |
| BMI | −.324* | .034 | .569** | .000 | −.346* | .025 |
| WHR | −.268** | .006 | .547** | .000 | −.564** | .000 |

Exclusion: Two women were excluded with insulin levels of 104 and 176, respectively.

TABLE No. 9b
Pearson Correlation Coefficients 2-Tailed Significance;
Non-Estrogen Users N = 117

| | | | | | Sex Hormone- | |
|---|---|---|---|---|---|---|
| | | | | | Binding | |
| | Estradiol | | Insulin | | Globulin | |
| | Pearson | Sig. 2- | Pearson | Sig. 2- | Pearson | Sig. 2- |
| Factor | correl. | tailed | correl. | tailed | correl. | tailed |
| Insulin | −.059 | .532 | | | −.378** | .000 |
| SHGB | .059 | .527 | −.378** | .000 | | |
| Estradiol | | | −.059 | .532 | .059 | .527 |
| E2 x SHBG | .830** | .000 | −.154 | .099 | .319** | .000 |
| Testosterone- | .065 | .489 | −.085 | .362 | .022 | 813 |
| total | | | | | | |
| TT/SHBG | .014 | .878 | .253** | .006 | −.473** | .000 |
| T-Free | .181 | .051 | −.002 | .979 | −.232* | .012 |
| Cortisol | .097 | .225 | −.146 | .117 | .060 | .523 |
| AndrosteneD | .156 | .093 | .076 | .448 | −.091 | .327 |
| DHEAS | .010 | .918 | −.113 | .226 | −.108 | .246 |
| Triglycerides | .032 | .740 | .250** | .008 | −.326** | .000 |
| Cholesterol | .101 | .287 | −.050 | .597 | −.062 | .516 |
| HDL- | −.069 | .470 | −.225* | .017 | .367** | .000 |
| HDL/C Ratio | −.127 | .181 | −.126 | .186 | .302** | .001 |
| LDL-Chol | .122 | .196 | −.028 | .769 | −.125 | .189 |
| Apo-B | .171 | .070 | .063 | .508 | −.139 | .142 |
| BP Systolic | −.159 | .116 | .192 | .058 | −.238* | .018 |
| BP Diastolic | .027 | .790 | .199 | .050 | −.203* | .044 |
| Age | −.335** | .000 | −.242** | .009 | .105 | .259 |
| BMI | .128 | .184 | .544** | .000 | −.397** | .000 |
| WHR | .003 | .976 | .265** | .004 | −.221* | .016 |

Exclusion: Two women were excluded with insulin levels of 104 and 176, respectively.

Pearson Correlation coefficients comparing variables between the group with estrogen replacement and those without estrogen appear in Table No. 10.

TABLE No. 10
Pearson Correlation coefficients: Cross Groups

| | estrogen | No estrogen | |
|---|---|---|---|
| | replacement | replacement | |
| | therapy | therapy | p value |
| Number of | 49 | 117 | |
| women | | | |
| Age | 57.8 ± 9.7 | 60.4 ± 11.4 | n.s. |
| Race | | | |
| ˜White | 40 (38.8%) | 63 (61.2%) | .001 |
| ˜Black | 7 (10.8%) | 58 (89.2%) | |
| sex hormone- | 217 ± 104 | 115 ± 60 | .001 |
| binding | | | |
| globulin (49) | | | |
| Estradiol | 107 ± 72 | 19.5 ± 41.2 | .001 |
| Insulin | 8.12 ± 5.8 | 12.4 ± 8.0 | .002 |
| Cortisol | 15.5 ± 7.3 | 12.8 ± 5.0 | .01 |
| bodymass | 28.3 ± 5.7 | 31.1 ± 7.0 | .02 |
| index | | | |
| Triglycerides | 147 ± 75 | 120 ± 68 | .03 |
| HDL | 60.0 ± 16 | 53.7 ± 14 | .03 |
| Cholesterol | | | |
| Dehydro- | 99.5 ± 64 | 102 ± 58 | n.s. |
| epiandro- | | | |
| sterone sulfate | | | |
| Testosterone | 0.337 ± 0.59 | 0.262 ± 0.21 | n.s. |
| Free Estradiol | 0.722 ± 0.61 | 0.560 ± 0.50 | n.s. |
| Total | 235 ± 30 | 227 ± 43 | n.s. |
| Cholesterol | | | |
| Chol/HDL ratio | 4.22 ± 1.2 | 4.46 ± 1.3 | n.s. |
| HDL/Chol ratio | 0.256 ± 0.073 | 0.245 ± 0.078 | n.s. |
| Apoprotein B | 117 ± 25 | 115 ± 31 | n.s. |
| Waist to Hip | 0.79 ± 0.067 | 0.83 ± 0.074 | .008 |

In Table No. 10, the cross correlation performed between the non-estrogen and estrogen replacement groups shows that estradiol and sex hormone-binding globulin are significantly higher and that waist-hip ratio and insulin levels are lower in the estrogen replacement therapy group (p<0.002). In the estrogen replacement therapy group, the body mass index is lower (p<0.01) while the cortisol, triglycerides and HDL-cholesterol are raised (p<0.03).

Cross correlation appears in Table No. 11. Independent and dependent variables in the equation appear in Table 11. Graphic displays of these cross group correlation appear in Scattergrams.

TABLE No. 11
Dependent variable. DISEASE (MI, HTN. DM, ANG)

| | |
|---|---|
| −2 Log likelihood | 214.55939 |
| ∘ constant is included in the model | |
| Variables entered on Step Number | |
| 1. Insulin | |
| HDLCHOL | |
| SINAIHDL | |
| Estradiol | |
| Testosterone | |
| sex hormone-binding globulin | |

Estimation terminated at iternation number 4 because Log likelihood decreased by less than 0.01 percent

| −2 Log likelihood | 189.698 | | |
|---|---|---|---|
| Goodness of fit | 172.253 | | |
| Cox and Snell −Rˆ 2 | 0.148 | | |
| Natgelkerke −Rˆ 2 | 0.198 | | |
| | Chi Square | dr | Significance |
| Model | 24.862 | 6 | .0004 |
| Block | 24.862 | 6 | .0004 |
| Step | 24.862 | 6 | .0004 |

Classification Table for DISEASE

The cut value is 0.50

Predicted

| | No | Yes | Percent correct |
|---|---|---|---|
| Observed No | 50 | 24 | 67.57% |
| Observed Yes | 28 | 53 | 65.43% |
| | Overall | 66.45% | |

Variables in the Equation: Logorithmic Regression

| Variable | B | S.E. | Wald | df | Sig | R | Exp(B) |
|---|---|---|---|---|---|---|---|
| Insulin | 0.0898 | 0.0290 | 9.5883 | 1 | 0.0020 | 0.1892 | 1.0940 |
| HDL/C | −1.2285 | 3.5175 | 0.1220 | 1 | 0.7269 | 0.0000 | 0.2927 |
| HDL | 0.0154 | 0.0184 | 0.7033 | 1 | 0.4017 | 0.0000 | 1.0155 |
| Estradiol | −0.0049 | 0.0029 | 2.8997 | 1 | 0.0886 | −.0658 | 0.9981 |
| Testos- | −0.7494 | 0.7129 | 1.1049 | 1 | 0.2932 | 0.0000 | 0.4727 |
| terone | | | | | | | |
| sex | −0.0028 | 0.0025 | 1.2267 | 1 | 0.2680 | 0.0000 | 0.9972 |
| hormone- | | | | | | | |
| binding | | | | | | | |
| globulin | | | | | | | |
| Constants | −1.0630 | 0.8682 | 1.1049 | 1 | 0.2208 | | |

Table No. 11 shows that only insulin is the independent variable that correlates with the presence of all disease states.

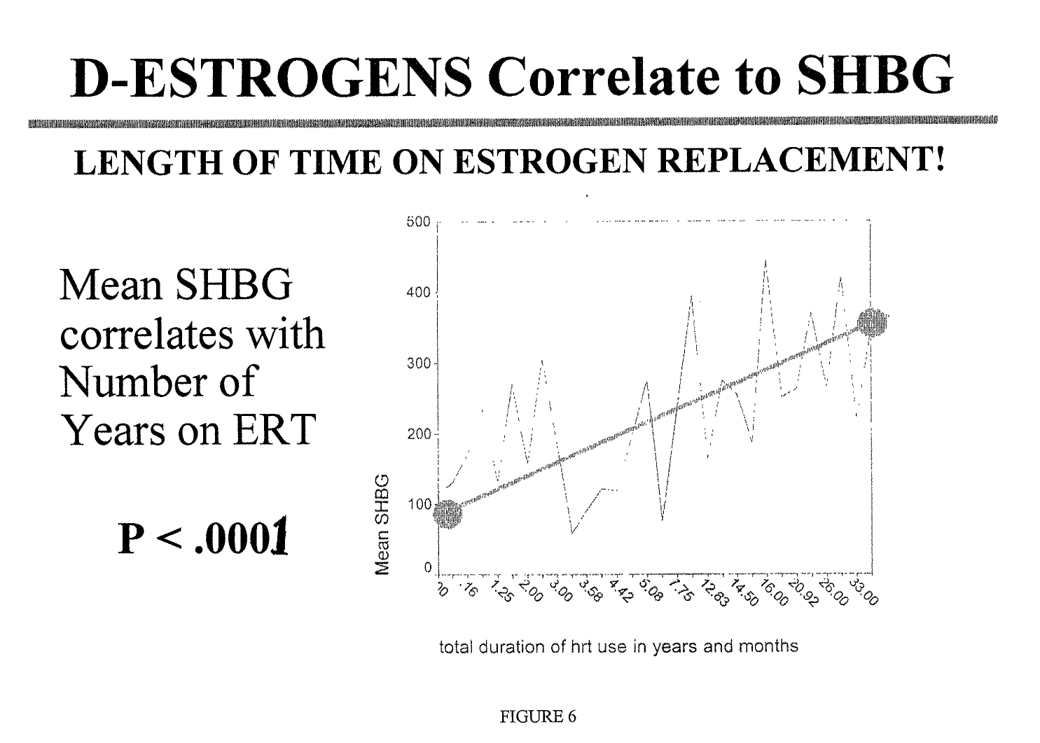

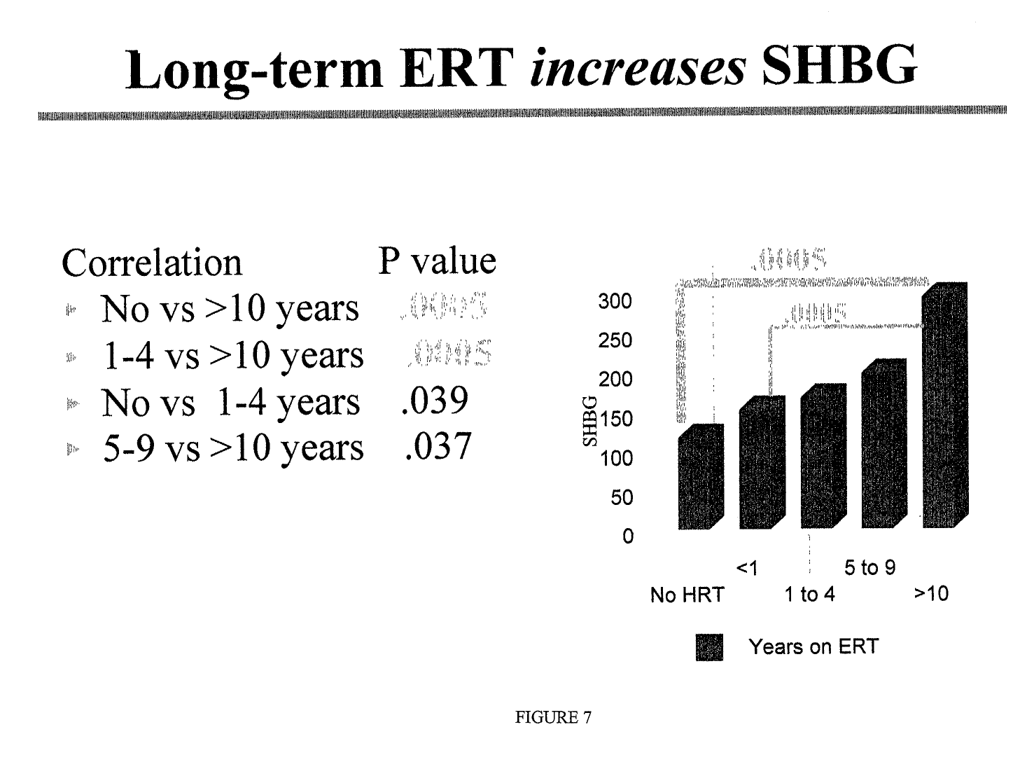

FIG. 6 shows the graph of the mean sex hormone-binding globulin versus insulin for current estrogen users. FIG. 7 shows the graphs of the mean sex hormone-binding globulin versus duration of time on estrogen replacement therapy (D-estrogen replacement therapy).

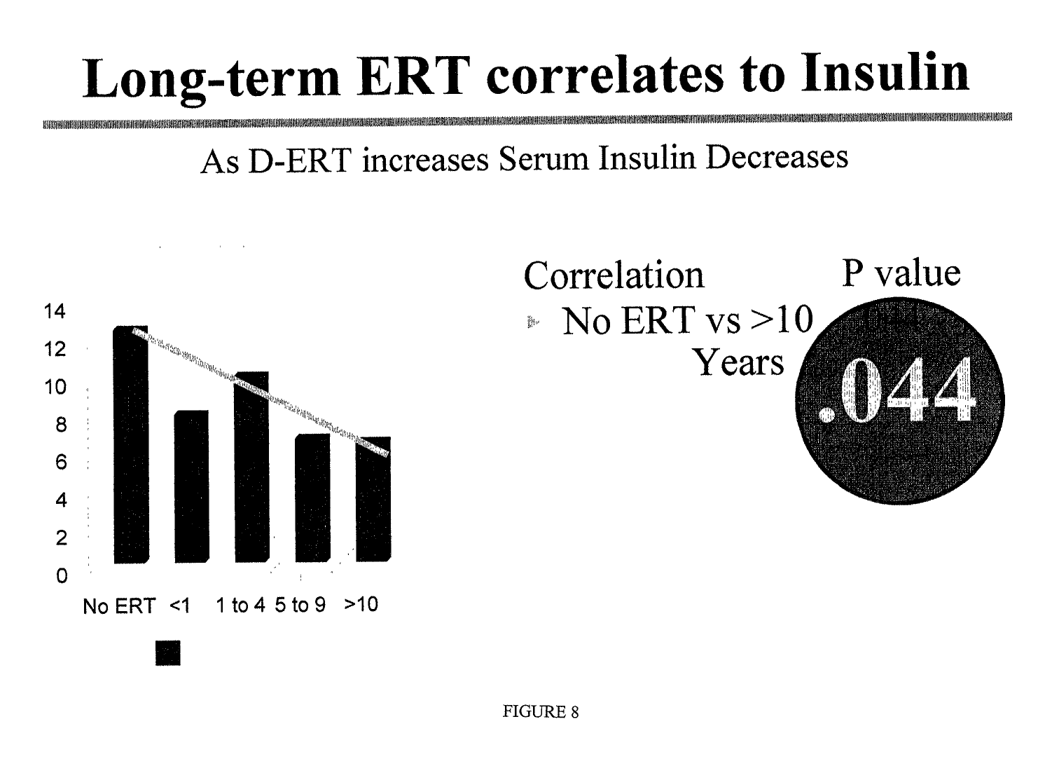

FIG. 8 shows the mean fasting insulin versus duration of time on estrogen replacement therapy (D-estrogen replacement therapy).

### Example 8

### Free Insulin Testosterone Factor in men

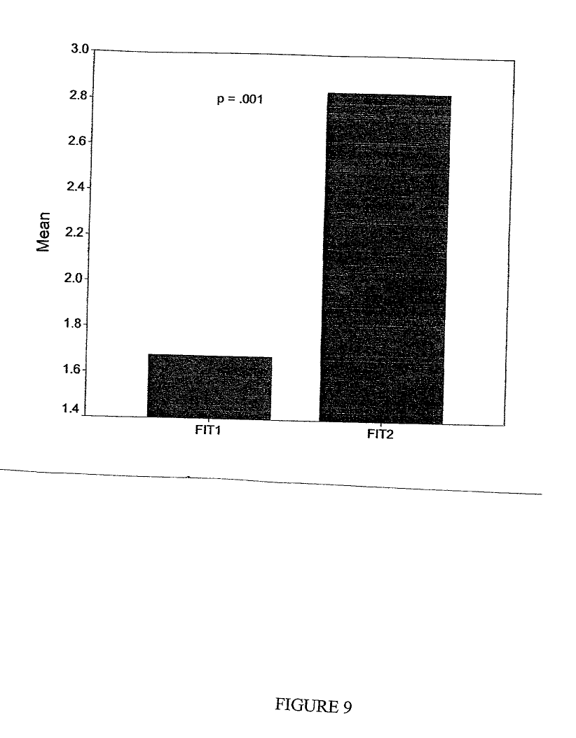

In the data set seen in the graph labeled FIG. 9, and the correlation in Table No. 12, the significance in 2-tailed paired samples is 0.001. In paired samples correlations the significance is also 0.0001. The men with the higher testosterone values and lower sex hormone-binding globulin have the greater F.I.T. factor.

TABLE No. 12
Correlation of Testosterone, Sex Hormone-Binding Globulin
Paired Samples Statistics

| | | | Std. | Std. Error |
|---|---|---|---|---|
| | Mean | N | Deviation | Mean |
| Pair 1 testosterone/insulin*shbg | 1.6779 | 13 | .8405 | .2331 |
| FIT2 | 2.8385 | 13 | 1.6810 | .4662 |

Paired Samples Correlations

| | N | Correlation | Sig |
|---|---|---|---|
| Pair 1 testosterone/insulin*shbg & | 13 | .891 | .000 |
| FIT2 | | | |

Paired Samples Test

| | Paired Differences | | | | | |
|---|---|---|---|---|---|---|
| | | | | 95% Confidence | | |
| | | | | Interval of the | | |
| | | Std. | Std. Error | Difference | | |
| | Mean | Deviation | Mean | Lower | Upper | t |
| Pair 1 | −1.1605 | 1.0066 | .2792 | −1.7688 | −.5522 | −4.157 |
| testosterone/insulin* shbg- | | | | | | |
| FIT2 | | | | | | |

Paired Samples Test

| | | Sig. |
|---|---|---|
| | df | (2-tailed) |
| Pair 1 testosterone/insulin*shbg- | 12 | .001 |
| FIT2 | | |

### Example 9

### Free Insulin Testosterone Factor in women

In the data set seen in Table No. 13, the correlation for non-estrogen users shows a correlation of the F.I.T. factor without testosterone (FIT2) or with the addition of testosterone (FIT3) to have a 2-tailed significance of 0.01 and 2-tailed significance of 0.000 in reference to age and thereafter disease state.

TABLE No. 13
Correlation for Non-Estrogen Users
Descriptive Statistics^{a}

| | | Std. | |
|---|---|---|---|
| | Mean | Deviation | N |
| AGE | 60.66 | 11.43 | 112 |
| FIT | 15.3311 | 16.3603 | 105 |
| FIT2 | 302.6817 | 968.2539 | 105 |
| FIT3 | 1298.2431 | 3944.2675 | 105 |
| FIT4 | 81.8687 | 95.8017 | 105 |

^{a}ERTUSE2 = 2

TABLE No. 13
Correlation for Non-Estrogen Users
Descriptive Statistics^{a}

| | | Std. | |
|---|---|---|---|
| | Mean | Deviation | N |
| AGE | 60.66 | 11.43 | 112 |
| FIT | 15.3311 | 16.3603 | 105 |
| FIT2 | 302.6817 | 968.2539 | 105 |
| FIT3 | 1298.2431 | 3944.2675 | 105 |
| FIT4 | 81.8687 | 95.8017 | 105 |

^{a}ERTUSE2 = 2

Correlations^{a}

| | | AGE | FIT | FIT2 | FIT3 | FIT4 |
|---|---|---|---|---|---|---|
| AGE | Pearson Correlation | | | | | |
| | Sig. (2-tailed) | | | | | |
| | N | | | | | |
| FIT | Pearson Correlation | .073 | | | | |
| | Sig. (2-tailed) | .460 | | | | |
| | N | 105 | | | | |
| FIT2 | Pearson Correlation | −.262** | .460** | | | |
| | Sig. (2-tailed) | .007 | .000 | | | |
| | N | 105 | 105 | | | |
| FIT3 | Pearson Correlation | −.261** | .376** | .894** | | |
| | Sig. (2-tailed) | .007 | .000 | .000 | | |
| | N | 105 | 105 | 105 | | |
| FIT4 | Pearson Correlation | .017 | .705** | .209* | .292** | |
| | Sig. (2-tailed) | .861 | .000 | .033 | .003 | |
| | N | 105 | 105 | 105 | 105 | |

***Correlation is significant at the 0.01 level (2-tailed)
*Correlation is significant at the 0.05 level (2-tailed)
^{a}ERTUSE2 = 2

Correlations

| | | | | | | |
|---|---|---|---|---|---|---|
| | | AGE | FIT | FIT2 | FIT3 | FIT4 |
| AGE | Pearson Correlation | | | | | |
| | Sig. (2-tailed) | | | | | |
| | N | | | | | |
| FIT | Pearson Correlation | .065 | | | | |
| | Sig. (2-tailed) | .408 | | | | |
| | N | 162 | | | | |
| FIT2 | Pearson Correlation | −.074 | .674** | | | |
| | Sig. (2-tailed) | .348 | .000 | | | |
| | N | 162 | 162 | | | |
| FIT3 | Pearson Correlation | −.001 | .542** | .864** | | |
| | Sig. (2-tailed) | .989 | .000 | .000 | | |
| | N | 162 | 162 | 162 | | |
| FIT4 | Pearson Correlation | .064 | .734** | .657** | .813** | |
| | Sig. (2-tailed) | .415 | .000 | .033 | .000 | |
| | N | 162 | 162 | 162 | 162 | |

**Correlation is significant at the 0.01 level (2-tailed)

In addition, it is intended that the present invention cover compounds made either using standard organic synthetic techniques, including combinatorial chemistry or by biological methods, such as through metabolism.

The contents of all cited references throughout this application are hereby expressly incorporated by reference. The practice of the present invention will employ, unless otherwise indicated, conventional techniques of pharmacology and pharmaceutics, which are within the skill of the art.

Although the invention has been described with respect to specific embodiments and examples, it should be appreciated that other embodiments utilizing the concept of the present invention are possible without departing from the scope of the invention. The present invention is defined by the claimed elements, and any and all modifications, variations, or equivalents that fall within the spirit and scope of the underlying principles.

What is claimed is:

1. A method for monitoring hormone concentration in a mammal, comprising:
(i) receiving a serum sample from the mammal;
(ii) measuring the concentration of insulin, estrogen, testosterone, and sex hormone-binding globulin in the serum; and
(iii) determining an F factor;
wherein determining the F factor for a female mammal is performed with the equation
F_{f}=(E×S×Q)/N;
wherein determining the F factor for a male mammal is performed with the equation
Fₘ=(T×P)/(S×N); and
wherein E is estrogen serum concentration; S is sex hormone-binding globulin serum concentration; Q is a conversion factor; N is fasting insulin serum concentration; T is testosterone serum concentration; and P is a conversion factor.

2. The method of claim 1, wherein Q is 0.02.

3. The method of claim 1, wherein P is equal to the product of 0.5 nmol/L times the fasting insulin serum concentration.

4. A method for treating a hormone disorder in a mammal, comprising:
(i) receiving a serum sample from the mammal;
(ii) measuring the concentration of insulin, estrogen, testosterone, and sex hormone-binding globulin in the serum;
(iii) determining an F factor; and
(iv) administering to the mammal a hormone disorder-effective amount of a therapeutic agent that comprises at least one of insulin, an estrogenic hormone, a steroid in the testosterone synthetic pathway, a sex hormone-binding globulin, an insulin inhibiting agent, an estrogen inhibiting agent, a testosterone inhibiting agent, a sex hormone-binding globulin inhibiting agent, a pharmaceutical agent that increases the serum concentration of estrogen, a pharmaceutical agent that increases the serum concentration of testosterone, a pharmaceutical agent that increases the serum concentration of sex hormone-binding globulin, and a pharmaceutical agent that increases the serum concentration of insulin in the mammal; and all salts, esters, amides, enantiomers, isomers, tautomers, prodrugs and derivatives thereof;
wherein determining the F factor for a female mammal is performed with the equation
F_{f}=(E×S×Q)/N;
wherein determining the F factor for a male mammal is performed with the equation
Fₘ=(T×P)/(S×N); and
wherein E is estrogen serum concentration; S is sex hormone-binding globulin serum concentration; Q is a conversion factor; N is fasting insulin serum concentration; T is testosterone serum concentration; and P is a conversion factor.

5. The method of claim 4, wherein the conversion factor Q is 0.02.

6. The method of claim 4, wherein the conversion factor P is equal to the product of 0.5 nmol/L times the fasting insulin serum concentration.

7. The method of claim 4, wherein the hormone disorder is associated with, or related to, a disease, wherein the disease comprises at least one of cardiovascular disease, Alzheimer's disease, dementia, cataracts, hypogonadism, sexual dysfunction, hypercholesterolemia, diabetic retinopathy, hyperinsulinemia, hyperglycemia, hypertension, obesity, osteoporosis, osteopenia, vaginal dryness, thinning of the vaginal wall, menopausal symptoms and hot flashes, cognitive dysfunction, and cancer.

8. The method of claim 7, wherein the cancer comprises at least one of cervical cancer, uterine cancer, and breast cancer.

9. The method of claim 4, wherein the therapeutic agent is administered once.

10. The method of claim 4, wherein the therapeutic agent is administered multiple times.

11. The method of claim 4, wherein the therapeutic agent is administered orally, percutaneously, transmucosally, by implantation, by inhalation, rectally, vaginally, topically, buccally, or parenterally.

12. The method of claim 11, wherein the buccal administration comprises sublingual administration.

13. The method of claim 11, wherein the parenteral administration comprises subcutaneous, intramuscular, intravenous, intramedullary, infusion, or intradermal administration.

14. The method of claim 4, wherein the mammal is a human.

15. The method of claim 14, wherein the insulin serum concentration decreases as compared to the concentration level before administration of the therapeutic agent.

16. The method of claim 14, wherein the human is female.

17. The method of claim 16, wherein the sex hormone-binding globulin serum concentration is increased as compared to the concentration level before administration of the therapeutic agent.

18. The method of claim 14, wherein the human is male.

19. The method of claim 18, wherein the sex hormone-binding globulin serum concentration is lowered as compared to the concentration level before administration of the therapeutic agent.

20. A method for treating a disorder associated with, or related to, a hormone disorder in a mammal, comprising:
(i) receiving a serum sample from the mammal;
(ii) measuring the concentration of insulin, estrogen, testosterone, and sex hormone-binding globulin in the serum;
(iii) determining an F factor; and
(iv) administering to the mammal a hormone disorder-effective amount of a therapeutic agent that comprises at least one of insulin, an estrogenic hormone, a steroid in the testosterone synthetic pathway, a sex hormone-binding globulin, an insulin inhibiting agent, an estrogen inhibiting agent, a testosterone inhibiting agent, a sex hormone-binding globulin inhibiting agent, a pharmaceutical agent that increases the serum concentration of estrogen, a pharmaceutical agent that increases the serum concentration of testosterone, a pharmaceutical agent that increases the serum concentration of sex hormone-binding globulin, and a pharmaceutical agent that increases the serum concentration of insulin in the mammal; and/or salts, esters, amides, enantiomers, isomers, tautomers, prodrugs and derivatives thereof;
wherein determining the F factor for a female mammal is performed with the equation
F_{f}=(E×S×Q)/N;
wherein determining the F factor for a male mammal is performed with the equation
Fₘ=(T×P)/(S×N); and
wherein E is estrogen serum concentration; S is sex hormone-binding globulin serum concentration; Q is a conversion factor; N is fasting insulin serum concentration; T is testosterone serum concentration; and P is a conversion factor.

21. The method of claim 20, wherein the conversion factor Q is 0.02.

22. The method of claim 20, wherein the conversion factor P is equal to the product of 0.5 nmol/L times the fasting insulin serum concentration.

23. The method of claim 20, wherein the disorder associated with, or related to, a hormone disorder comprises at least one of cardiovascular disease, Alzheimer's disease, dementia, cataracts, hypogonadism, sexual dysfunction, hypercholesterolemia, diabetic retinopathy, hyperinsulinemia, hyperglycemia, hypertension, obesity, osteoporosis, osteopenia, vaginal dryness, thinning of the vaginal wall, menopausal symptoms and hot flashes, cognitive dysfunction, and cancer.

24. The method of claim 23, wherein the cancer comprises at least one of cervical cancer, uterine cancer, and breast cancer.

25. The method of claim 20, wherein the therapeutic agent is administered once.

26. The method of claim 20, wherein the therapeutic agent is administered multiple times.

27. The method of claim 20, wherein the therapeutic agent is administered orally, percutaneously, transmucosally, by implantation, by inhalation, rectally, vaginally, topically, buccally, or parenterally.

28. The method of claim 27, wherein the buccal administration comprises sublingual administration.

29. The method of claim 27, wherein the parenteral administration comprises subcutaneous, intramuscular, intravenous, intramedullary, infusion, or intradermal administration.

30. The method of claim 20, wherein the mammal is a human.

31. The method of claim 30, wherein the insulin serum concentration decreases as compared to the concentration level before administration of the therapeutic agent.

32. The method of claim 30, wherein the human is female.

33. The method of claim 32, wherein the sex hormone-binding globulin serum concentration is increased as compared to the concentration level before administration of the therapeutic agent.

34. The method of claim 30, wherein the human is male.

35. The method of claim 34, wherein the sex hormone-binding globulin serum concentration is lowered as compared to the concentration level before administration of the therapeutic agent.

36. A method for restoring hormone balance in a mammal in need thereof comprising:
(i) receiving a serum sample from the mammal;
(ii) measuring the concentration of insulin, estrogen, testosterone, and sex hormone-binding globulin in the serum;
(iii) determining an F factor; and
(iv) administering to the mammal a hormone disorder-effective amount of a therapeutic agent that comprises at least one of insulin, an estrogenic hormone, a steroid in the testosterone synthetic pathway, a sex hormone-binding globulin, an insulin inhibiting agent, an estrogen inhibiting agent, a testosterone inhibiting it agent, a sex hormone-binding globulin inhibiting agent, a pharmaceutical agent that increases the serum concentration of estrogen, a pharmaceutical agent that increases the serum concentration of testosterone, a pharmaceutical agent that increases the serum concentration of sex hormone-binding globulin, and a pharmaceutical agent that increases the serum concentration of insulin in the mammal; and salts, esters, amides, enantiomers, isomers, tautomers, prodrugs and derivatives thereof;
wherein determining the F factor for a female mammal is performed with the equation
F_{f}=(E×S×Q)/N;
wherein determining the F factor for a male mammal is performed with the equation
Fₘ=(T×P)/(S×N); and
wherein E is estrogen serum concentration; S is sex hormone-binding globulin serum concentration; Q is a conversion factor; N is fasting insulin serum concentration; T is testosterone serum concentration; and P is a conversion factor.

37. The method of claim 36, wherein the conversion factor Q is 0.02.

38. The method of claim 36, wherein the conversion factor P is equal to the product of 0.5 nmol/L times the fasting insulin serum concentration.

39. The method of claim 36, wherein the disorder associated with, or related to, a hormone disorder comprises at least one of cardiovascular disease, Alzheimer's disease, dementia, cataracts, hypogonadism, sexual dysfunction, hypercholesterolemia, diabetic retinopathy, hyperinsulinemia, hyperglycemia, hypertension, obesity, osteoporosis, osteopenia, vaginal dryness, thinning of the vaginal wall, menopausal symptoms and hot flashes, cognitive dysfunction, and cancer.

40. The method of claim 39, wherein the cancer comprises at least one of cervical cancer, uterine cancer, and breast cancer.

41. The method of claim 36, wherein the therapeutic agent is administered once.

42. The method of claim 36, wherein the therapeutic agent is administered multiple times.

43. The method of claim 36, wherein the therapeutic agent is administered orally, percutaneously, transmucosally, by implantation, by inhalation, rectally, vaginally, topically, buccally, or parenterally.

44. The method of claim 43, wherein the buccal administration comprises sublingual administration.

45. The method of claim 43, wherein the parenteral administration comprises subcutaneous, intramuscular, intravenous, intramedullary, infusion, or intradermal administration.

46. The method of claim 36, wherein the mammal is a human.

47. The method of claim 36, wherein the insulin serum concentration decreases as compared to the concentration level before administration of the therapeutic agent.

48. The method of claim 36, wherein the human is female.

49. The method of claim 48, wherein the sex hormone-binding globulin serum concentration is increased as compared to the concentration level before administration of the therapeutic agent.

50. The method of claim 36, wherein the human is male.

51. The method of claim 50, wherein the sex hormone-binding globulin serum concentration is lowered as compared to the concentration level before administration of the therapeutic agent.

52. A kit for treating a hormone disorder in a mammal, comprising: a hormone disorder-effective amount of a therapeutic agent, the therapeutic agent comprising at least one of insulin, an estrogenic hormone, a steroid in the testosterone synthetic pathway, a sex hormone-binding globulin, an insulin inhibiting agent, an estrogen inhibiting agent, a testosterone inhibiting agent, a sex hormone-binding globulin inhibiting agent, a pharmaceutical agent that increases the serum concentration of estrogen, a pharmaceutical agent that increases the serum concentration of testosterone, a pharmaceutical agent that increases the serum concentration of sex hormone-binding globulin, and a pharmaceutical agent that increases the serum concentration of insulin in the mammal; and salts, esters, amides, enantiomers, isomers, tautomers, prodrugs and derivatives thereof.

53. The kit of claim 52, wherein the hormone disorder is associated with, or related to, a disease, the disease comprising at least one of cardiovascular disease, Alzheimer's disease, dementia, cataracts, hypogonadism, sexual dysfunction, hypercholesterolemia, diabetic retinopathy, hyperinsulinemia, hyperglycemia, hypertension, obesity, osteoporosis, osteopenia, vaginal dryness, thinning of the vaginal wall, menopausal symptoms and hot flashes, cognitive dysfunction, and cancer.

54. The method of claim 52, wherein the cancer comprises at least one of cervical cancer, uterine cancer, and breast cancer.

55. The kit of claim 52, wherein the therapeutic agent is administered once.

56. The kit of claim 52, wherein the therapeutic agent is administered multiple times.

57. The kit of claim 52, wherein the therapeutic agent is administered orally, percutaneously, transmucosally, by implantation, by inhalation, rectally, vaginally, topically, buccally, or parenterally.

58. The kit of claim 57, wherein the buccal administration comprises sublingual administration.

59. The kit of claim 57, wherein the parenteral administration comprises subcutaneous, intramuscular, intravenous, intramedullary, infusion, or intradermal administration.

60. The kit of claim 52, wherein the mammal is a human.

61. The kit of claim 60, wherein the insulin serum concentration decreases as compared to the concentration level before administration of the therapeutic agent.

62. The kit of claim 60, wherein the human is female.

63. The kit of claim 62, wherein the sex hormone-binding globulin serum concentration is increased as compared to the concentration level before administration of the therapeutic agent.

64. The kit of claim 52, wherein the human is male.

65. The kit of claim 64, wherein the sex hormone-binding globulin serum concentration is lowered as compared to the concentration level before administration of the therapeutic agent.
